(19) United States
(12) Patent Application Publication
(10) Pub. No.: US 2010/0262185 A1
(43) Pub. Date: Oct. 14, 2010

(54) Method and apparatus for aperture fixation by securing flexible material with a knotless fixation device

(75) Inventors: Jeffrey Gelfand, Severna Park, MD (US); Robert Collins, Annapolis, MD (US); Christina Morganti, Severna Park, MD (US); Stephen Santangelo, Shelton, CT (US)

Correspondence Address: Steven DeSmet;Leahy & DeSmet, LLC, Suite 900, 11785 Beltsville Drive, Calverton, MD, 20705, US

(73) Assignee: Suspension Orthopaedic Solutions, LLC, Annapolis, MD (US)

(21) Appl. No.: 12/662,336

(22) Filed: Apr. 12, 2010

Related U.S. Application Data
(60) Provisional application No. 61/212,407, filed on Apr. 10, 2009.

(51) Int. Cl. A61B 17/04

(52) U.S. Cl. 606/232

(57) ABSTRACT

A knotless aperture fixation system, and method for use thereof, for securing a flexible material (i.e., suture, cable, fiber tape, or any other suitable flexible material) to a nearby tissue using knotless fixation achieved by a compression fit of the flexible material between two smooth surfaces. The system comprises a cylindrical or spherical flanged sleeve with threaded upper portion and smooth tapered lower portion is used in conjunction with a headless set screw with a bullet-shaped tip to achieve a compression fit of the flexible material between the smooth tapered lower portion of the sleeve and the smooth bullet-shaped tip of the headless set screw. The system may be used in conjunction with an expanded washer with a recessed lip, a flanged washer, or a bone plate with an opening containing a recessed lip.

### CROSS-REFERENCE TO RELATED APPLICATIONS

The present application derives priority from U.S. provisional application No. 61/212,407 filed Apr. 10, 2009.

### BACKGROUND OF THE INVENTION

1. Field of the Invention

The present invention relates broadly to the field of surgery, and, more particularly to a method of aperture fixation that uses knotless fixation to secure suture or other flexible material in connection with tissue reconstructions or fracture repairs.

2. Description of the Background

There is often a need in surgery to secure tissue, such as a tendon, ligament, or bone under tension to a neighboring tissue (e.g., another bone or bone surface). Suture or other flexible material (i.e., cable, fiber tape, or any other suitable flexible material) can be used to secure tissue (e.g. ligament, tendon, bone) to a nearby bone or other medical device such as a bone plate or expanded washer resting on the surface of a nearby bone. This flexible material will often be tensioned between the neighboring tissue (at one end) and a bone or medical device at the other at the other end. This tension will often be maintained by securing the suture or flexible material to the neighboring tissue with a fastening device or method (such as an anchor into the bone, a button secured with suture through the bone, or a weave of suture or other flexible material with tendon), and, at the other end, by securing the flexible material to a bone or medical device. On the end secured to the medical device, securing the suture or flexible material to a bone, bone plate, screw, button or other suitable fixation device typically requires the use of a knot in the suture or flexible material at the location where the suture or flexible material is secured. There are, however, often instances where it is desirable to utilize a method of securing suture or other flexible material that does not require the use of a knot. For instance, when the fastening device is inaccessible, as inside a joint; or when the fastening device is located in a subcutaneous position such that the bulk of the knot may create symptoms of pain and/or skin irritation. There may also be situations in which a knot may not maintain the appropriate tension on the flexible material demanded by the surgical procedure. Certain knotless solutions have been conceived in the past, such as U.S. Pat. No. 7,090,690, however, the knotless fixation methods described therein typically rely on engagement between suture or other flexible material and the threads of a screw, anchor or similar device (i.e., creating a zig-zag shaped binding lock on the flexible material as the flexible material is forced into the tortuous path created by the engaged threads). Such reliance on thread engagement creates a significant risk of abrasion of the flexible material against the threading of the screw or other device. The present invention addresses these concerns by taking a fundamentally different approach to the engagement of the suture or flexible material. In brief, the present invention comprises a partially tapered sleeve and matching partially tapered (headless) set partially tapered (headless) set screw. In the absence of any flexible material passing through the sleeve, the sleeve and set screw engage each other with a loose and sloppy fit. When flexible material has been passed through the sleeve prior to insertion of the headless set screw, the fit becomes firm and tight as the flexible material is secured with a compression fit between the smooth tapered distal inner surface of the sleeve and the smooth tapered distal outer surface of the headless set screw as the screw is advanced in the sleeve. By intentionally designing the system to reduce any reliance for fixation on the tortuous path of the flexible material through the threads, the risk of abrasion is substantially reduced.

There are numerous surgical procedures for which such a method or system of securing tissue under tension to a neighboring tissue with a flexible material is advantageous and desirable, but for which no good solution currently exists. One example of such a surgical procedure is the treatment of an injured AC Joint. At present, treatment options for AC Joint injuries include: primary ACJ fixation (with pins, screws, suture between buttons, bone plates (such as a clavicle hook plate)) with or without ligament repair or reconstruction; primary coracoclavicular interval fixation (with a coracoclavicular screw, wire, fascia, conjoint tendon, or synthetic sutures) with or without incorporation of AC or CC ligament repair/reconstruction; excision of the distal clavicle with or without coracoclavicular ligament repair with fascia or suture, or coracoacromial ligament transfer; dynamic muscle transfers with or without excision of the distal clavicle. There are several complications associated with these current methodologies including: Metal implants can migrate post surgery, can cause other complications, and, in many cases, require a second surgical procedure for removal of the metal implants; muscle transfers are technically demanding and technically demanding and cannot address dislocation of the ACJ; significant potential exists for injury to the musculocutaneous nerve; loss of screw fixation or screw breakage; or flexible material failure which may occur from abrasion of flexible material on bone edges or screw threads, buttons or other similar devices.

A recent example of such a method of AC Joint repair is described in U.S. patent application Ser. No. 11/687,882, Acromioclavicular Joint Fixation Technique (the “AJFT Patent”). This method secures suture between two button devices and creates tension in the suture between the two buttons. At each end, the suture passes through a button as described in detail in the AJFT Patent. The suture is then secured at the superior end with a knot. Because this method requires the use of a knot in a subcutaneous position, complications are possible (as described above). Further, because the suture is tensioned between two buttons, with the suture passing through a drilled bone tunnel in two separate bones, the possibility of abrasion of the suture against the rough edges on the superior and inferior aspects of each bone is present because of the movement of the shoulder girdle. Finally, manipulating the buttons on the inferior side of an unexposed or partially exposed bone can be cumbersome.

Another, similar injury without a good current solution is a fracture of the distal clavicle. Current treatments rely, among other things, on similar surgical procedures to the ones described above for AC Joint injuries (and can result in similar complications). In addition, surgeons use specialized bone plates designed for distal clavicle fractures. Despite attempts to customize these plates to address the complexities of, and biomechanical forces related to, these injuries, studies have shown complications relating to, among other things, the lack of screw purchase in the injured bone.

The present invention comprises a method of knotless fixation that eliminates the need for a knot to secure the flexible material (i.e., suture, cable, fiber tape, or any other suitable flexible material) to the medical device (e.g., washer, bone plate). Further, the method of aperture fixation of the present invention, through the compression fit between two smooth surfaces as further described herein below and the provision of a smooth channel through the bone with smooth rounded edges, both reduces the reliance on screw purchase for the screws in the bone plate and mitigates the possibility of abrasion of the flexible material against the threading of screws or other devices as well as against the raw cut edges of the bone itself. In addition to the indications described above, the present invention has broad applicability to a multitude of other injuries to the body in areas that are sensitive to the placement of knots, that lack the ability for strong purchase by bone screws or other fixation methods, and in which suture (or other flexible material) abrasion is a potential issue or complication. Examples of other indications for which the present invention represents a marked departure from current devices include, but are not limited to, ACL injuries, distal biceps repairs, ulna fractures, and other ligament injuries about the elbow, ankle and knee.

### SUMMARY OF THE INVENTION

The present invention is a knotless aperture fixation system for securing a flexible material material (i.e., suture, cable, fiber tape, or any other suitable flexible material) to a nearby tissue using knotless fixation achieved by a compression fit of the flexible material between two smooth surfaces. There are many surgical procedures in which it is desirable to use suture or another flexible material to create tension between a tissue at one end and a bone or rigid medical device at the other end. In these situations, one end of the flexible material may be secured to such neighboring tissue with a bone anchor into an adjacent bone, a button on the inferior side of an adjacent bone secured with suture, a weave of suture or other flexible material with a nearby tendon, or another appropriate method. At the other end, it is desirable to secure the suture or flexible material to the bone or rigid medical device in a low-profile, knotless manner. The sleeve and headless set screw described in the present invention, either alone or in conjunction with an expanded washer or customized bone plate, accomplishes this goal.

In a preferred embodiment, the fastening device consists of a partially internally threaded sleeve (the threads extend for approximately the upper two-thirds of the interior of the sleeve) with a substantially round flange on the proximal end of the sleeve and a tapered distal end to the sleeve that mates with a headless set screw wherein the distal tip of the headless set screw resembles the shape of a bullet. The lower portion of the threaded sleeve with flange contains no threads and narrows (is tapered inward) toward the distal end of the sleeve. The taper in the sleeve is designed to match the bullet-shaped rounded tip of the headless set screw. The threaded sleeve and headless set screw are made of stainless steel, titanium, PEEK, or other suitable biocompatible material. The flange of the threaded sleeve is substantially round and may or may not possess two flat edges opposite and parallel to each other. The upper portion of the sleeve immediately beneath the immediately beneath the flange also may or may not possess two flat edges that correspond to the flat edges of the flange, again opposite and parallel to each other. The flat edges of the flange allow for the flange and sleeve to seat securely on the recessed shelf of an opening in a bone plate or on the recessed shelf of an expanded washer. The flat edges on the flange and the flat edges on the upper portion of the sleeve provide the sleeve with additional stability and resistance to rotation of the sleeve within the opening of the bone plate or washer when the headless set screw is being inserted into the sleeve and tightened. In some embodiments, engagement with an appropriate tensioning device provides the resistance to rotation.

In another embodiment, the flange of the threaded sleeve and the sleeve itself are round, without the “flats” described above. The flange will be substantially larger in this embodiment to permit the sleeve with flange to sit directly on cortical bone, rather than inside a bone plate or washer.

In another embodiment, the sleeve is essentially round and has substantially the shape of a sphere with a cylindrical opening that passes straight through the spherical sleeve. The interior of the cylindrical opening contains threads on the upper portion of the sleeve and when the threads end, the interior of the cylindrical opening is smooth and tapers inward so that the cylindrical opening is a smaller diameter at the smooth end than at the threaded end. Where the smooth end of the cylindrical opening ends at other end of the sherical sleeve, the edges are smooth and rounded to minimize abrading of the flexible material that is to be passed through the sleeve. The bullet-shaped headless set screw as described will be used in the spherical sleeve in essentially the same manner as described to secure flexible material that passes through the spherical sleeve. The spherical sleeve. The set screw is seated within the sleeve and tightened to the appropriate torque with flexible material present, which will then both maximize the resistance to the flexible material slipping through the matching reciprocal surfaces at the tapered portion of the sleeve and resist pullout of the set screw. This spherical sleeve will be used in conjunction with a flanged washer that is of a diameter larger than that of the spherical sleeve. The flanged washer will be substantially round with a flat perimeter that will rest on the surface of a bone. Inside the flat perimeter of the flanged washer the flanged washer is recessed downward leading to an opening at the bottom of the washer. The recess inside the flat perimeter of the flanged washer has a smooth surface and resembles the shape of a bowl and the opening at the bottom of the washer will be substantially round in some embodiments while in other embodiments the opening will be elongated.

The spherical sleeve and flanged washer are utilized when there is a need to use a knotless fixation method to secure a tissue through a hole drilled through a bone and the line of tension that needs to be maintained subtends an angle that is not substantially perpendicular to the surface of the bone on which the washer rests. In some embodiments the spherical sleeve may have features on its surface to engage an appropriate tensioning device that will allow it to resist rotation as the headless set screw is inserted.

The headless set screw has a rounded tip and is designed and sized to seat loosely (e.g., a “sloppy fit”) within the threaded portion of the sleeve when no flexible material is passed through the sleeve. This design element reduces the abrasion caused by the tortuous path of the flexible material through the threads of the sleeve and set screw. When flexible material is passed through passed through the sleeve and the set screw is fully screwed into (threaded into) the sleeve, the rounded bullet-shaped tip of the set screw engages the suture or other flexible material between the smooth tip of the set screw and the smooth tapered end at the distal end of the sleeve in a compression fit. The headless set screw is seated within the sleeve and tightened to the appropriate torque with suture or other flexible material present, which both maximizes the resistance to the suture or other flexible material slipping through the matching reciprocal smooth surfaces at the tapered portions of the sleeve and headless set screw and resists pullout of the headless set screw.

The interior of the sleeve contains an upper threaded portion and a lower narrower, tapered smooth portion. The distal end of the sleeve, which may or may not extend out the opposite end of the bone through which it is passed, is smooth and rounded to prevent abrasion of the suture or other flexible material which is passed through the sleeve. The threaded upper portion of the interior of the sleeve extends deep enough into the sleeve to permit the headless set screw to be inserted into the sleeve so that the proximal end of the headless set screw is buried beneath the surface at the proximal end of the threaded sleeve with flange and the distal end of the set screw with its rounded tip fits snugly against the tapered surface of the narrower lower smooth portion of the sleeve thereby applying pressure to and securing the flexible material to maintain the tension on the suture or flexible material. Ideally, a torque-limiting driver is utilized to insert the headless set screw to ensure proper torque that will maximize resistance of slippage of the flexible material through the tapered surfaces. Securing the suture or flexible material in this way is beneficial because it creates uniform compression on the suture or flexible material between the tapered between the tapered surface and the bullet shaped headless set screw tip and avoids reliance on the threads of the sleeve and the headless set screw for fixation. Additionally, this virtually eliminates the concern that the threads of the sleeve and screw will damage or abrade the suture or flexible material because the compression that secures the suture or flexible material occurs at the distal end of the headless set screw and distal to the threads of both the headless set screw and sleeve interior. In some instances the surgeon may also choose to tie a knot in the flexible material above the headless set screw in which case the knot could be pressed into the upper portion of the sleeve above the fully seated set screw such that the knot is recessed below the proximal surface of the threaded sleeve with flange. The threaded sleeve with flange will most often be inserted through a washer or bone plate and into a predrilled hole in cortical bone, wherein the predrilled hole is of a diameter greater than the sleeve but lesser than the diameter of the flange of the threaded sleeve with flange. The flange of the threaded sleeve is wide enough to prevent the sleeve from falling into or through the extended washer, an opening in a bone plate, or a hole drilled through cortical bone. In some embodiments, the flange of the threaded sleeve is wide enough to sit directly on the cortical bone without the use of a washer or bone plate, and in such an embodiment the underside of the flange may or may not be contoured to more closely resemble the surface of the bone atop which it will be placed.

In another embodiment, after the headless set screw has been fully inserted into the threaded sleeve with flange, a separate cap may be placed in the opening atop the set screw to prevent the possibility of the screw backing out. Alternatively, the surgeon may elect to tie a knot in the flexible material above the set screw to prevent backing out. While the compression fit fit between of the headless set screw within the sleeve is sufficient to prevent the headless set screw from backing out, these measures may be taken as a fail safe.

In one procedure using the invention (the “Described Procedure”), a bone fracture or ligament injury can be repaired by securing suture or another flexible material at one end to a nearby tissue by means of a bone anchor, button, weave of suture or other flexible material to tendon, or other appropriate method. The other end of the suture or flexible material is secured by the sleeve and set screw of the present invention. The suture or flexible material can be secured to nearby tissue in a variety of ways (as noted above). In the case of a bone anchor or button, suture or other flexible material may be pre-attached to a fastening device (such as a pre-loaded anchor driver or button-deployment assembly). This fastening device would typically include a handle, cannulated driver shaft and connection to either an anchor or button. The suture or flexible material would be pre-attached to the anchor or button, then fed through the cannulated driver shaft. In some instances the flexible material may be doubled over to increase the aggregate strength of the flexible material. Typically, as part of the procedure, the fastening device assembly is passed through a hole that has been drilled in a bone (either in the fractured bone or through a bone near the ligament injury) and the bone anchor, button or other appropriate device, is secured to a tissue opposite the hole in the bone opening at the distal end (either deployed on the inferior side of such other tissue if a button is used or screwed into such other tissue if a bone anchor is used). Alternatively, the suture or flexible material could be woven into a nearby tendon (rather than securing the suture or other flexible material to an anchor or button to a nearby tissue and either retrieving or passing it through the hole through the bone).

The free end or ends of the suture or other flexible material (i.e., the end or ends of the suture or flexible material that is not attached to the bone anchor, button or tendon) extends up through the hole created in the bone as described above. The free end(s) of the flexible material is then passed through the sleeve, and the sleeve is moved along the suture or other flexible material until it is secured in the hole in the bone. While maintaining tension on the suture or flexible material, the set screw is inserted, locking the suture or other flexible material in place.

In most embodiments of the present invention, a bone plate or washer will sit on the superior aspect of the bone through which the sleeve passes. Such washer or bone plate has an opening that sits atop the hole in the bone through which the flexible material has been passed. An example of a bone plate for this type of application is described in US Patent Application Number 2010/0016899. In such case, the procedure would be performed in the same manner as described above; however, before being passed through the sleeve, the flexible material is passed through such bone plate or washer, then through the sleeve. The sleeve is then seated in the bone plate or washer as described below.

When the threaded sleeve with flange is used in conjunction with a bone plate or extended washer, the threaded sleeve with flange is passed through an elongated opening in the bone plate or a substantially round opening through an extended washer wherein the flange is seated securely within said opening. An example of a bone plate for this type of application is described in US Patent Application Number 2010/0016899. The engagement of the sleeve with the bone plate or washer is more fully described in the detailed drawings. When the opening in the bone plate is elongated the flat edges of the flange serve to lock the threaded sleeve in position from side to side position from side to side within the elongated opening. Additionally, the elongated opening in the bone plate possesses a shelf around the entire inside perimeter of the opening wherein the shelf extends inward from the edge of the elongated opening to provide for a secure seating of the flange at any location within the elongated opening and for the flange of the sleeve to sit flush with the superior surface of the bone plate. Similarly, the washer possesses a shelf around the entire inside perimeter of the center opening of the washer to provide for a secure seating of the flange within the center opening.

As an example, the present invention provides a method of knotless fixation for the treatment of clavicle fractures or AC Joint injuries. For a clavicle fracture (especially a distal clavicle fracture), the method comprises securing a substantially rigid plate, most likely of the type described in US Patent Application Number 2010/0016899, to the superior aspect of the clavicle so that it extends along the clavicle on either side of the fracture. Once the plate is in position on the superior aspect of the clavicle or the fractured clavicle is provisionally reduced, a targeting method (such as the use of intraoperative fluoroscopy) can be used to target the coracoid process through the elongated slot. In this embodiment the coracoid process is the site for the fixation of the flexible material opposite the clavicle. A bone drill is then utilized to form a hole of appropriate size to accommodate the sleeve through the clavicle connecting the superior and inferior surfaces of the clavicle. A bone drill is also utilized to form a hole of appropriate size to accommodate the fastening device through the coracoid process connecting the superior and inferior surfaces of the coracoid process. The order of drilling the holes through the clavicle and the coracoid process is left to the surgeon's choice. The order of drilling the holes is not important important so long as the end result is that there are appropriate sized holes drilled through both the clavicle and the coracoid process. As described above in the Described Procedure, a fastening device (i.e., an anchor or button) with flexible material pre-attached is inserted through the hole drilled in the clavicle via a cannulated coracoid bone anchor driver assembly or button deployment assembly. The suture or flexible material is then secured to the coracoid process by screwing the anchor into the superior aspect of the coracoid process or by inserting the assembly through the coracoid process and deploying the button on the inferior side of the coracoid process. The cannulated coracoid bone anchor driver assembly or button deployment assembly is then removed, leaving the flexible material exposed and extending up through the hole through the clavicle (and through the hole in the bone plate if one has already been affixed to the fractured clavicle). The threaded sleeve with flange is placed over the flexible material (i.e., the flexible material is threaded through the middle of the sleeve), through the opening in the bone plate, and into the pre-drilled hole through the clavicle from superior to inferior. The suture or flexible material is then appropriately tensioned to restore the normal coracoclavicular interval. While tension is maintained, the headless screw is inserted into the threaded sleeve with flange (with flexible material already passing through) and is threaded onto the threads of the sleeve until the rounded tip of the screw engages the flexible material between the rounded tip of the screw and the tapered portion of the inside of the sleeve, thereby securing the tension on the flexible material with a knotless, press-fit technique. Intraoperative fluoroscopy (or other suitable procedure) can be utilized to confirm the location of the fastening device (i.e., the anchor, button or other suitable device) and to confirm the anatomical reduction and restoration of the normal anatomical interval. of the normal anatomical interval.

Similarly, the treatment of high grade AC Joint injuries would be performed in substantially the same manner as described for the clavicle fractures above; however, instead of using the bone plate described, such bone plate could be replaced with the washer described above. Alternatively, no washer is used and the sleeve is placed directly into the hole in the clavicle with the flange of the sleeve resting directly on the superior surface of the clavicle.

The use of the threaded sleeve that passes completely through the bone and is anchored to the coracoid process with the flexible material mitigates the potential for axial pull-out of bone screws which is seen with other currently used fixation methods that rely solely on screw purchase in a distal fragment. Additionally, use of the threaded sleeve and knotless fixation methods disclosed, whereby the sleeve passes at least substantially through if not completely through the bone, creates a smooth channel and eliminates the ability for the suture or flexible material to come into contact with sharp bone edges which may abrade the suture or flexible material and thereby minimizes the possibility of suture abrasion which has been reported with use of the fixation described in U.S. Patent Application Publication No. 2007/0179531, the disclosure of which is hereby incorporated herein by reference in its entirety.

### BRIEF DESCRIPTION OF THE DRAWINGS

FIG. 1 is an overhead angled side view of the threaded sleeve with flange.

FIG. 2 is an overhead angled side view of a cross-section of the threaded sleeve with flange.

FIG. 3 is an overhead view of a washer.

FIG. 4 is an overhead angled side view of the threaded sleeve with flange and washer separated.

FIG. 5 is an overhead angled side view of the threaded sleeve with flange and washer with the threaded sleeve with flange inserted through the opening in the washer.

FIG. 6 is an overhead angled side view of a cross-section of the threaded sleeve with flange and washer with the threaded sleeve with flange inserted through the opening in the washer.

FIG. 7 is an overhead angled side view of a bullet-shaped headless set screw.

FIG. 8A is an overhead angled side view of a cross-section of the threaded sleeve with flange with a loop of flexible material passing through the sleeve and a bullet-shaped headless set screw ready to be inserted into the sleeve and FIG. 8B is an overhead angled side view of a cross-section of the threaded sleeve with flange with a loop of flexible material passing through the sleeve and a bullet-shaped headless set screw fully inserted into the sleeve and securing a flexible material with a compression fit between the smooth tapered section of the sleeve and the smooth bullet-shaped section of the headless set screw.

FIG. 9 is an overhead view of a proprietary bone plate.

FIG. 10A is a side view of an anchor with an eyelet at its proximal end and FIG. 10B is an overhead view of the top of an anchor with an eyelet at its proximal end.

FIG. 11 is a partial perspective view of the bones of the anatomy of a human shoulder.

FIG. 12 is a partial perspective view of the bones of the anatomy of a human shoulder depicting a fractured clavicle.

FIG. 13 is a partial perspective view of the bones of the anatomy of a human shoulder depicting a high grade separation of the acromioclavicular joint.

FIG. 14 is a partial perspective view of the bones of the anatomy of a human shoulder with a fractured clavicle illustrating the exploded view of the elements of a knotless aperture fixation method.

FIG. 15 is a partial perspective view of the bones of the anatomy of a human shoulder with a fractured clavicle illustrating the tensioned elements of a knotless aperture fixation method.

FIG. 16 is a partial perspective view of the bones of the anatomy of a human shoulder with a dislocated clavicle illustrating the exploded view of the elements of a knotless aperture fixation method.

FIG. 17 is a partial perspective view of the bones of the anatomy of a human shoulder with a dislocated clavicle illustrating the tensioned elements of a knotless aperture fixation method.

FIG. 18 is a partial perspective view of the bones of the anatomy of a human knee illustrating the exploded view of the elements of a knotless aperture fixation method.

FIG. 19 is a partial perspective view of the bones of the anatomy of a human knee illustrating the tensioned elements of a knotless aperture fixation method.

### DETAILED DESCRIPTION OF THE PREFERRED EMBODIMENTS

The present application is directed to a system and method for aperture fixation. The system and method may be utilized to treat tissue injuries and bone fractures such as reconstructive procedures of a high grade acromioclavicular joint separation, the anterior cruciate ligament of the knee, biceps tendon, or the ulnar collateral ligament of the elbow or thumb. The system may also be used in conjunction with another device (e.g., a bone plate) to treat bone fractures (e.g., clavicle fractures).

In general, the present invention involves a knotless fixation method whereby a flexible material (such as suture) is secured by a compression fit between a partially threaded, tapered sleeve and a partially threaded, bullet-shaped, headless set screw. The interior of the threaded sleeve has a threaded upper portion which extends approximately two-thirds of the way down the sleeve from the proximal to the distal end, and then transitions to a smooth tapered lower portion. The partially threaded, bullet-shaped, headless set screw is similarly threaded approximately two-thirds of the way down the screw from the proximal end to the distal end, and then transitions to a smooth, tapered, “bullet-shaped” tip.

In a typical procedure using the present invention, a hole would be drilled through a bone, suture (or other flexible material) would be secured to tissue opposite such bone, and that suture (or other flexible material) would be passed through the hole in the bone from distal to proximal. The loose ends of the flexible material are passed through a bone plate or washer (as illustrated in the drawings) and the partially threaded sleeve. Once passed through the sleeve the flexible material is tensioned to restore the natural anatomical interval. While maintaining the tension on the flexible material the bullet-shaped headless screw is inserted fully into the threaded sleeve with flange so that the flexible material is secured between the smooth rounded tip of the headless screw and the smooth tapered lower portion of the sleeve. The engagement of the headless screw and the sleeve is such that the threads are used to engage the headless screw with the sleeve but are not the means to engage or hold the flexible material passed through the sleeve. Rather, the “press fit” between the smooth tapered tip of the set screw and the smooth tapered end of the sleeve holds the flexible material in place and tensioned. Although the flexible material may take a “tortuous path” as described in U.S. Pat. No. 7,090.690, such path is not necessary for the securing of the flexible material and, in fact, the abrasion resulting from such path is minimized. Through the smooth tapered fit and the smooth rounded edges on the distal end of the sleeve, the present invention addresses the risk of flexible material abrasion or severing as a result of contact with the threads of the set screw as well as from rubbing against the rough bone at the edges hole in the bone.

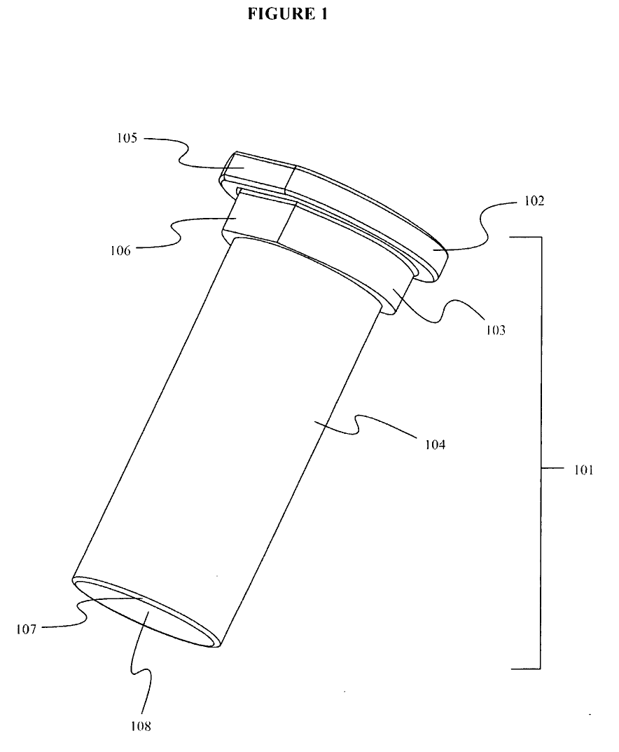

FIG. 1 is an overhead angled side view of the threaded sleeve with a flange 101. The figure illustrates that the exterior of the shaft of the sleeve 104 has a smooth rounded surface. Also illustrated in the figure is the flange 102 of the threaded sleeve with flange 101 which extends outward from the top perimeter of the shaft of the sleeve 104 a distance sufficient to prevent the threaded sleeve with flange 101 from falling through a hole in a bone, the washer 301, bone plate 901 or other device, when the threaded sleeve with flange 101 is inserted into a hole in a bone the washer 301, bone plate 901, or other device as part of an aperture fixation or fracture fixation methodology. The flange 102 as shown is essentially round with two flat sides 105 parallel to each other, which flat sides 105 may fit securely into the opening in a washer 301 or bone plate 901 in essentially a lock and key type of fit. Immediately beneath the flange 102 in this embodiment is the expanded upper portion 103 of the shaft of the sleeve 104 which is also essentially round and has two flat sides 106 parallel to each other and in line with the flat sides 105 of the flange and serves to enhance the lock and key type of fit. The locking type fit prevents rotation of the threaded sleeve with flange 101 when inserting the headless set screw 701. Also shown is the bottom opening 108 of the shaft of the sleeve 104 which is narrower than the top opening of the threaded sleeve with flange 101 and has a smooth non-threaded surface. The lip 107 of the bottom opening 108 of the shaft of the sleeve 104 has a smooth rounded edge to reduce the likelihood that any flexible material 1401 that may rub against the smooth rounded bottom lip 107 of the shaft of the sleeve 104 will abrade. Not shown in this Figure is the interior of the shaft of the sleeve 104 wherein the top portion of the interior of the shaft of the sleeve 104 contains threads 201. The threads 201 on the interior of the shaft of the sleeve 104 do not extend the entire length of the shaft of the sleeve 104, rather the threads 201 extend for only approximately the top two-thirds of the interior of the shaft of the sleeve 104. Where the threads 201 in the shaft of the sleeve 104 end the interior of the sleeve tapers in toward the bottom of the sleeve and it is between this taper and the smooth, distal end of the headless set screw 701 that the flexible material that is passed through the sleeve is secured.

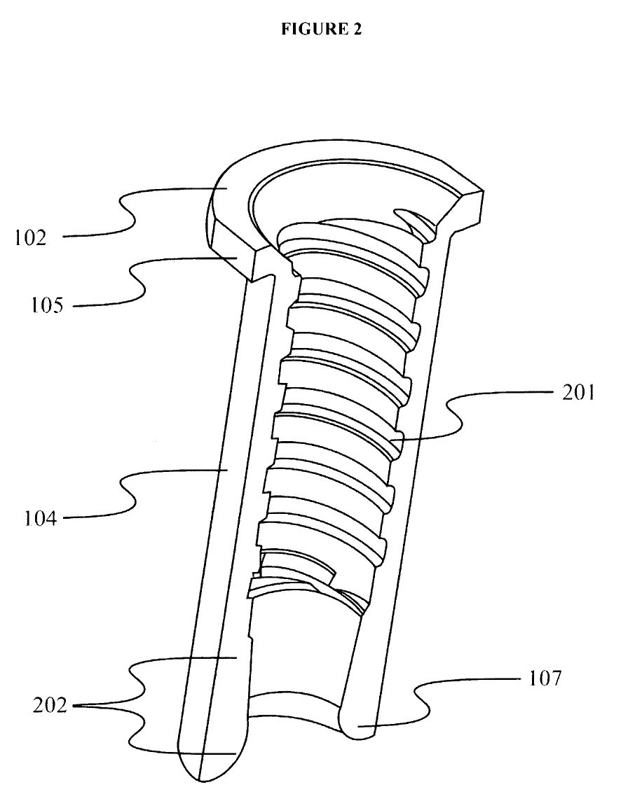

FIG. 2 is an overhead angled side view of a cross-section of the threaded sleeve with flange, illustrating the surface structure of the interior of the threaded sleeve with flange 101. The figure illustrates the smooth surface of the exterior of the shaft of the sleeve 104 as well as the flange 102 of the shaft of the sleeve 104 which extends outward from the top perimeter of the shaft of the sleeve 104 a distance sufficient to prevent the threaded sleeve with flange 101 from falling through a hole in a bone, the washer 301, bone plate 901, or other device as part of an aperture fixation or fracture fixation methodology. The cross-section is taken directly down the center of the threaded sleeve with flange 101 through the parallel flat sides 105 of the flange 102. This cross-section illustrates the entire length of the interior of the shaft of the sleeve 104. The threads 201 on the interior of the shaft of the sleeve 104 do not extend the entire length of the interior of the shaft of the sleeve 104, rather the threads 201 are only present in approximately the upper two-thirds of the interior of the shaft of the sleeve 104. At the point where the threads 201 stop on the interior of the shaft of the sleeve 104 the interior sides of the sleeve narrow to create a tapered effect 202 so that approximately the bottom one-third of the interior of the shaft of the sleeve 104 is narrower than approximately the top two-thirds of the interior of the shaft of the sleeve 104. The interior surface of the tapered effect 202 is smooth. It is between this smooth portion with the tapered effect 202 and the bullet-shaped, headless set screw 701 that the flexible material 1401 is secured. The press fit of the flexible material 1401 between the tapered, smooth surfaces reduces the risk of severing or abrading the flexible material 1401 that could otherwise occur if the flexible material 1401 is engaged between the actual threads 201 of the shaft of the sleeve 104 and threads 706 of the headless set screw 701. This figure also more clearly illustrates the smooth rounded bottom lip 107 of the shaft of the sleeve 104 which reduces abrasion of the flexible material 1401 utilized in an aperture fixation or fracture fixation methodology because the flexible material 1401 will not rub against the distal edge of the hole cut in the bone. In some cut in the bone. In some embodiments, as illustrated in this figure the threaded sleeve with flange 101 may not include an expanded upper portion 103 with flat sides 106 as shown in FIG. 1.

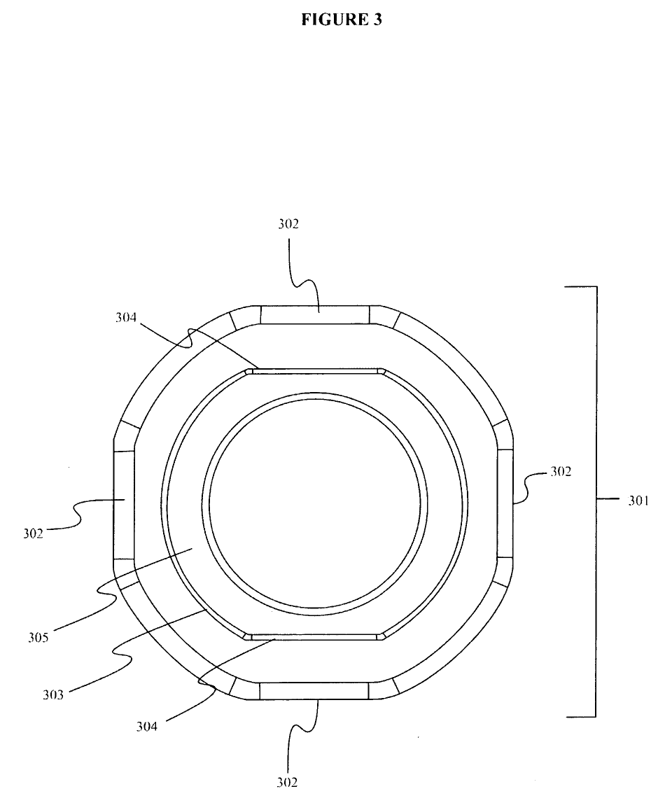

FIG. 3 is an overhead view of a washer illustrating the shape and structure of the washer 301 through which the threaded sleeve with flange may be passed through to increase the surface area at the surface of the bone and, in some cases, to prevent the threaded sleeve with flange from falling through a hole drilled through the bone. In one embodiment, the washer 301 as illustrated is essentially round with four flattened sides 302 positioned opposite each other. The inner perimeter 303 of the washer 301 is essentially round with two flattened sides 304. The shape of the inner perimeter 303 of the washer 301 mirrors the shape of the flange 102 on the threaded sleeve with flange 101 so as to create a type of lock and key fit between the flange 102 of the threaded sleeve with flange 101 and the washer 301 such that the top of the sleeve will sit flush with the top of the washer. Additionally, as illustrated, the inner perimeter 303 of the washer 301 has a recessed level 305 atop which the flange 102 of the threaded sleeve with flange 101 rests when the threaded sleeve with flange 101 is inserted into the washer 301. Although not depicted in this figure, the bottom surface of the washer may either be essentially flat or alternatively, in some embodiments, the bottom surface of the washer may be contoured so as to fit more snugly against the surface of the bone atop which the washer will set.

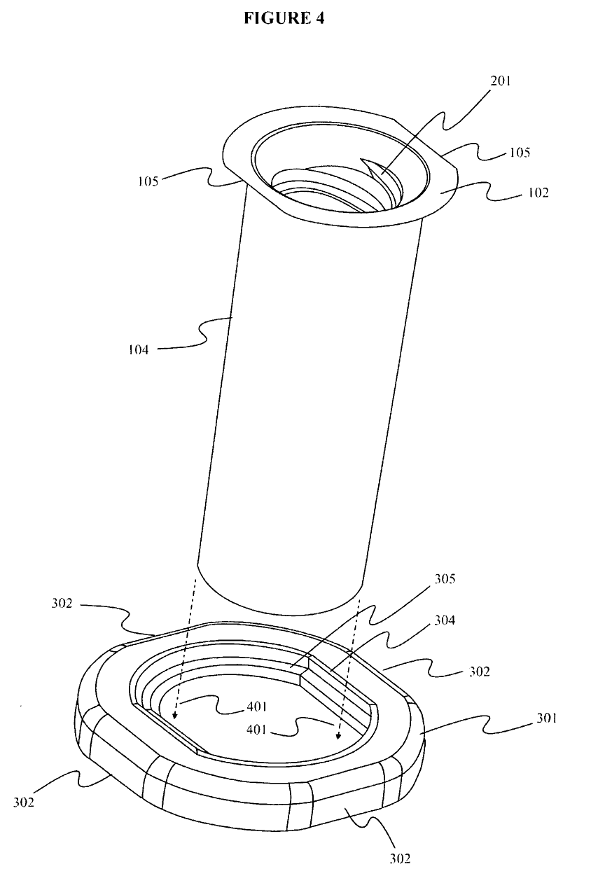

FIG. 4 is an overhead angled side view of the threaded sleeve with flange 101 and the washer 301 illustrating the threaded sleeve with flange 101 and washer 301 separated but aligned with one another. The arrows 401 depict the orientation with which the threaded sleeve with flange 101 is to be passed through the washer 301. The figure illustrates that the flattened sides 105 of the perimeter of the flange 102 of the threaded sleeve with flange 101 are aligned with the flattened sides 304 of the inner perimeter 303 of the washer 301 so that when the threaded sleeve with flange 101 is inserted into the washer 301 the flattened sides 105 of the flange 102 and the flattened sides 304 of the inner perimeter 303 of the washer 301 fit together and essentially lock the flange 102 of the threaded sleeve with flange 101 in place within the washer 301 and prevent the threaded sleeve with flange 101 from rotating.

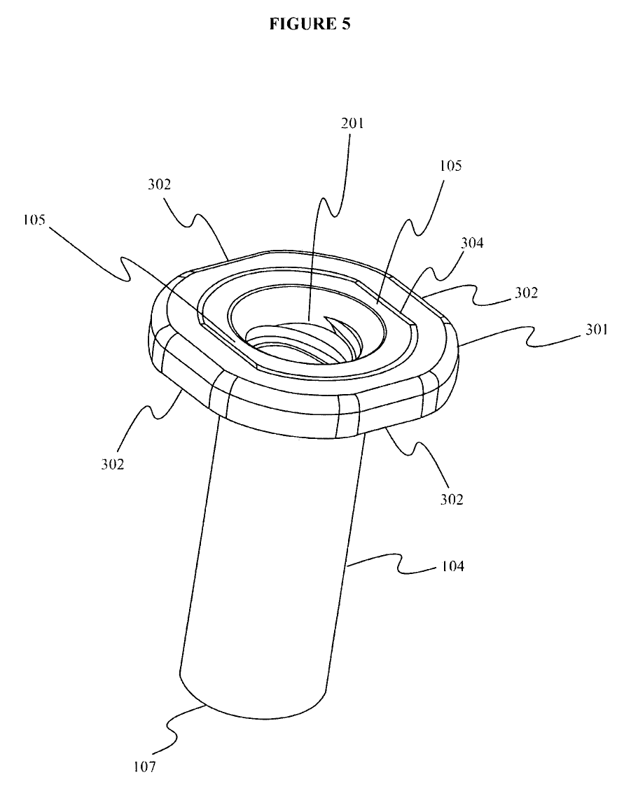

FIG. 5 is an overhead angled side view of the threaded sleeve with flange and washer illustrating the threaded sleeve with flange 101 fully inserted into the washer 301. As illustrated, when the threaded sleeve with flange 101 is fully inserted into the washer 301 the flange 102 of the threaded sleeve with flange 101 is essentially level (flush) with the surface of the washer 301.

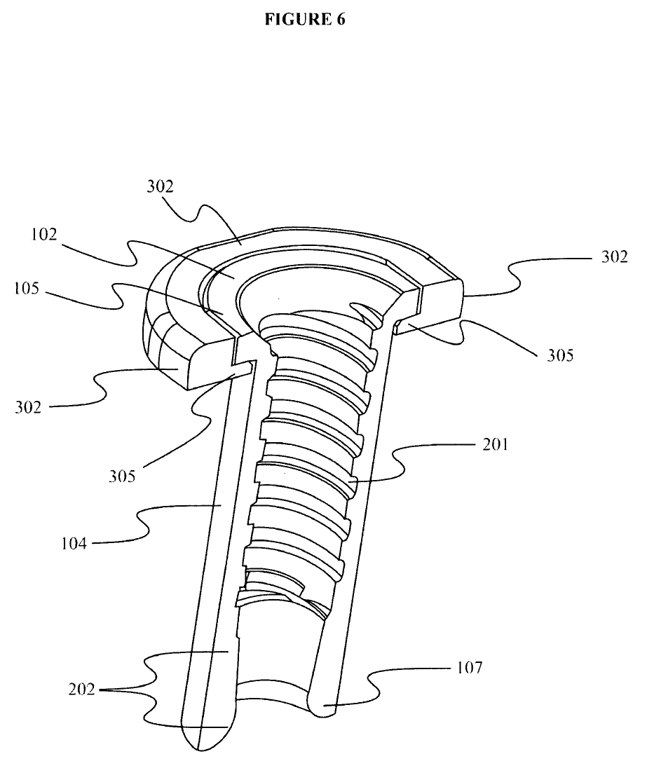

FIG. 6 is an overhead angled side view of a cross-section of the threaded sleeve with flange and washer, illustrating the recessed lip 305 on the interior perimeter 303 of the washer 301. The cross-section is taken directly down the center of the washer 301 and the threaded sleeve with flange 101 through the parallel flat sides 105 of the flange 102. The illustration shows how the recessed lip 305 prevents the flange 102 of the threaded sleeve with flange 101 from falling or passing all the way through the washer 301. The flange 102 of the threaded sleeve with flange 101 rests atop the recessed lip 305 of the washer 301 and resists passing completely through the washer 301. Additionally, the figure illustrates how the flattened sides 105 of the flange 102 are aligned with the flattened sides 304 of the washer 301 to lock the threaded sleeve with flange 101 in position and prevent the threaded sleeve with flange 101 from rotating. rotating.

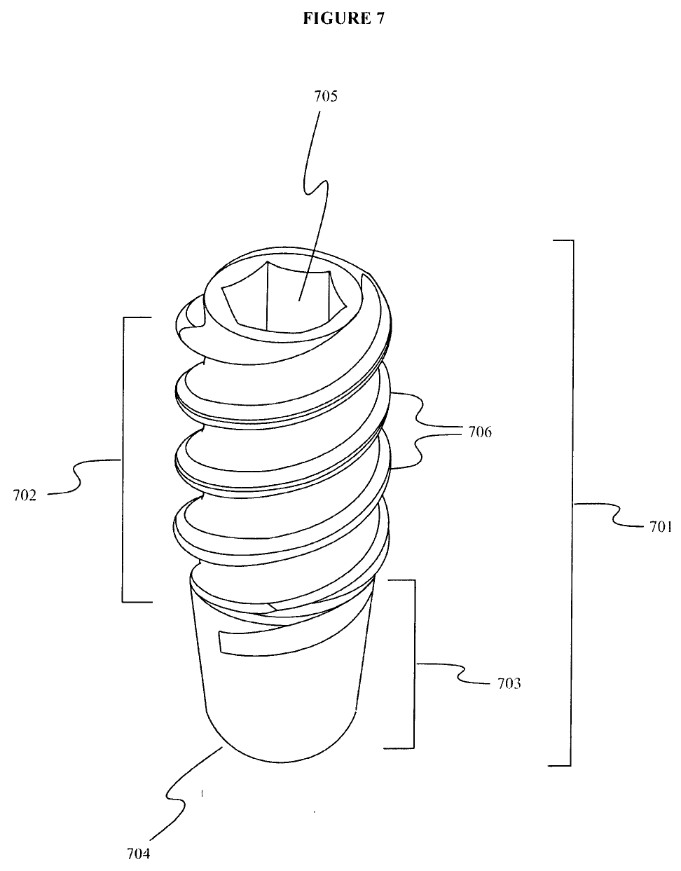

FIG. 7 is an overhead angled side view of the bullet-shaped headless set screw 701, illustrating upper threaded portion 702 and the lower smooth tapered portion 703. The figure also illustrates the blunt bullet-shaped tip 704 of the headless set screw 701, as well as the threads 706 of the upper threaded portion 702 of the headless set screw 701. The threads 706 of the headless set screw 701 are designed to engage the threads 201 of the threaded sleeve with flange 101 with a loose “sloppy” fit. In some embodiments the threads 706 of the headless set screw 701 are double threaded to facilitate quicker engagement of the threads and to permit faster insertion of the headless set screw with fewer rotations required. When a flexible material 1401 is passed through the threaded sleeve with flange 101 prior to insertion of the headless set screw 701 the loose sloppy fit becomes firm and tight as the flexible material is secured with a compression fit between the smooth tapered effect 202 of the threaded sleeve with flange 101 and the lower smooth tapered portion 703 and bullet-shaped tip 704 of the headless set screw 701 as the headless set screw 701 is advanced into the threaded sleeve with flange 101. Also illustrated is the engaging means 705. As illustrated the engaging means 705 of the headless set screw 701 is a hexagonal socket cavity formed in the superior surface of the headless set screw 701. This is only a single example of the many potential engaging means 701 that may be utilized on the headless set screw 701. The engaging means 705 may be of any form suitable for driving insertion of the headless set screw 701.

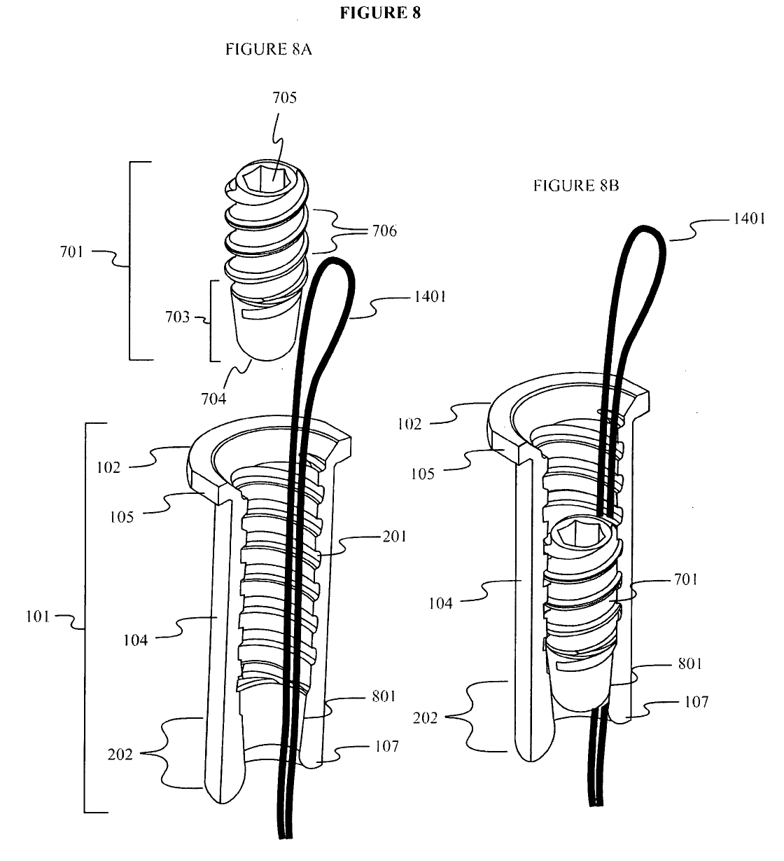

FIG. 8 contains two illustrations, FIG. 8A and FIG. 8B, and these figures illustrate the fit of the headless set screw 701 in the threaded sleeve with flange 101. FIG. 8A illustrates an an overhead angled side view of a cross-section of the threaded sleeve with flange 101 with a loop of flexible material 1401 passing up through the threaded sleeve with flange 101 and the headless set screw 701 positioned above the threaded sleeve with flange 101. As shown, the loop of flexible material 1401 is set off to the side of the interior of the threaded sleeve with flange 101. The location for the compression fit 801 is shown on the smooth tapered lower portion 202 within the interior of the threaded sleeve with flange 101. FIG. 8B illustrates the headless set screw 701 fully engaged into the threaded sleeve with flange 101 with the flexible material 1401 passing through the threaded sleeve with flange 101. The compression fit 801 occurs between the smooth tapered lower portion 202 within the interior of the threaded sleeve with flange 101 and the lower smooth tapered portion and bullet-shaped tip 703 & 704 of the headless set screw. Also illustrated is the smooth rounded bottom lip 107 of the shaft of the sleeve 104 which will protect the flexible material 1401 from abrasion.

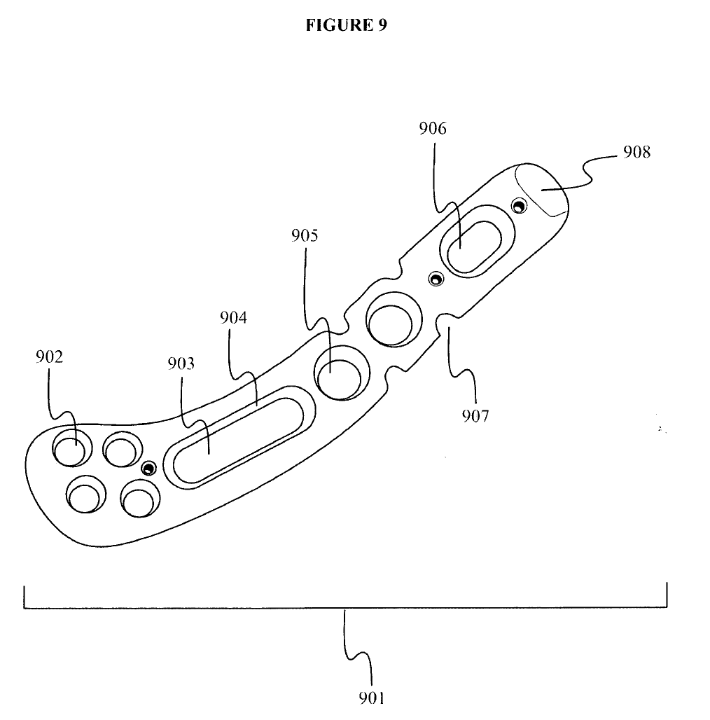

FIG. 9 is an overhead view of a proprietary bone plate similar to that described in U.S. Patent Application Number 2010/0016899. The figure illustrates a proprietary bone plate 901 for use in clavicle fracture fixation. As illustrated, the bone plate 901 has a plurality of small round openings 902 near the distal end of the plate and a plurality of larger round openings 905 nearer the center of the plate 901 and extending toward the medial end of the plate 901. The plate 901 also has a first elongated opening 903 in the plate that possesses a recessed lip 904, and a second elongated opening 906 in the plate 901 that does not have a lip or rim, but rather, is an adjustment slot with tapered sides so that a screw may be added while permitting the plate 901 to be adjusted slightly. The recessed lip 904 permits the engagement of a device such as the threaded sleeve with threaded sleeve with flange 101 without falling through the bone plate 901, and provides a solid surface against which forces can be applied to accomplish aperture fixation. The flange 102 of the threaded sleeve with flange 101 rests atop the recessed lip 904 so that the flattened sides 105 of the flange 102 sit against the inside sides of the elongated opening 903 in the plate 901. In some embodiments, the threaded sleeve with flange 101 may have an extended upper portion 103 with two flat sides 106 immediately beneath the flange 102 so that these flat sides 106 of the extended upper portion 103 will sit securely against the sides of the recessed lip 904 in the first elongated opening 903 of the plate 901. The flat sides 105 of the flange 102 engage on the sides with the sides of the elongated opening 903 of the plate 901 above the recessed lip 904. The flat sides 106 of the extended upper portion 103 of the shaft of the sleeve 104 press against the inner sides of the recessed lip 904 of the elongated opening 903 in the plate 901. The seating of the flange 102 against the sides of the first elongated opening 903 prevents rotation of the threaded sleeve with flange 101 when inserting the headless set screw 701 into the threaded sleeve with flange 101.

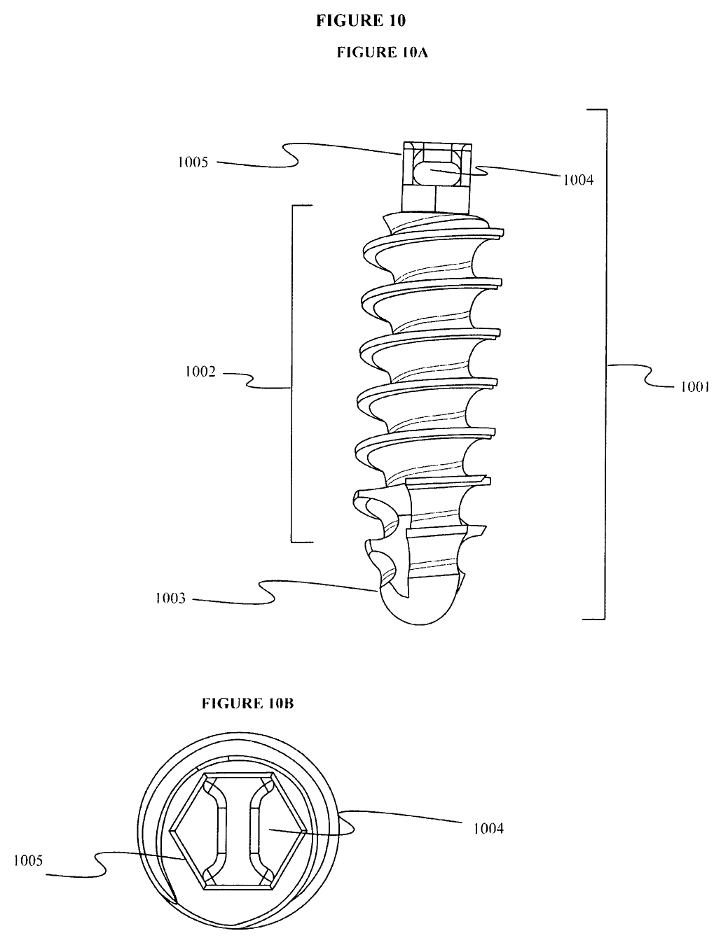

FIGS. 10A & 10B are a side view and top view of an anchor with an eyelet at its proximal end. FIG. 10A illustrates an anchor 1001 which may be inserted into a bone adjacent to the hole drilled through a bone to accommodate the threaded sleeve with flange 101. In some embodiments, the anchor 1001 may be inserted into the adjacent bone so that the entire length of the anchor 1001 passes through the bone and the tip 1003 of the anchor 1001 extends minimally beyond the distal end of the adjacent bone into which the anchor 1001 has been inserted. In other embodiments the anchor 1001 does not pass completely through the adjacent bone. The anchor 1001 as illustrated possesses threads 1002 along substantially the entire length of the anchor 1001 with only the tip 1003 of the anchor 1001 not possessing threads. In a typical embodiment, the tip 1003 of the anchor 1001 will be self-tapping. As illustrated, the anchor 1001 has an eyelet 1004 atop its proximal end. The eyelet 1004 is utilized to secure a flexible material to the anchor 1001. As illustrated, the eyelet 1004 has an overall perimeter shape 1005 that is hexagonal and is ideal to fit a pre-loaded anchor driver assembly. FIG. 10B is the top view of an anchor illustrating the hexagonal overall perimeter shape 1005 of the eyelet 1004.

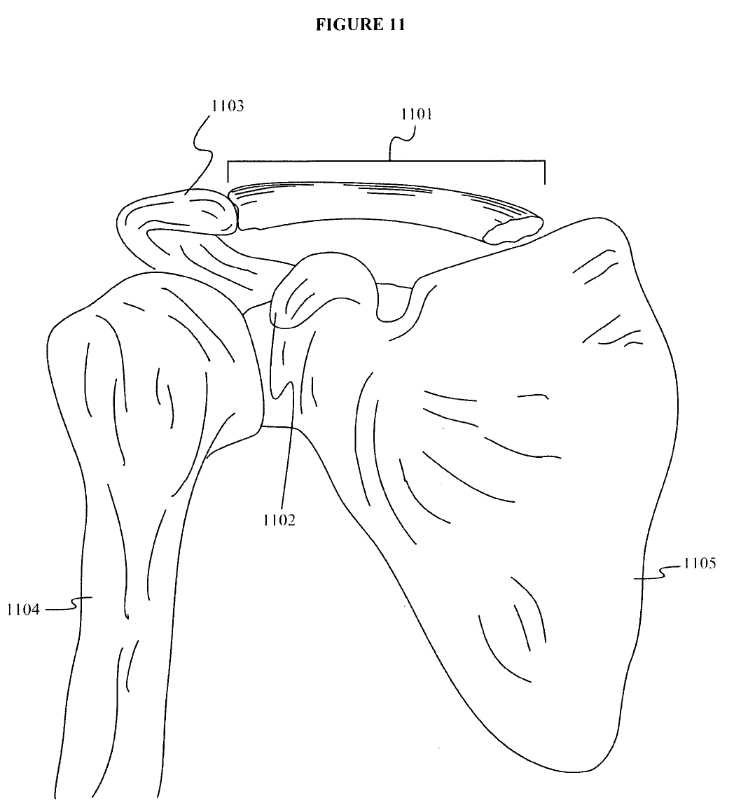

FIG. 11 is a partial perspective view of the bones of the normal shoulder anatomy. The figure illustrates the clavicle 1101, the coracoid process 1102, the acromion 1103, the body of the scapula 1105, and the humerus 1104. The coracoid process 1102 extends anteriorly from the scapula 1105. As the illustration shows, the coracoid process 1102 is located at least partially beneath the clavicle 1101.

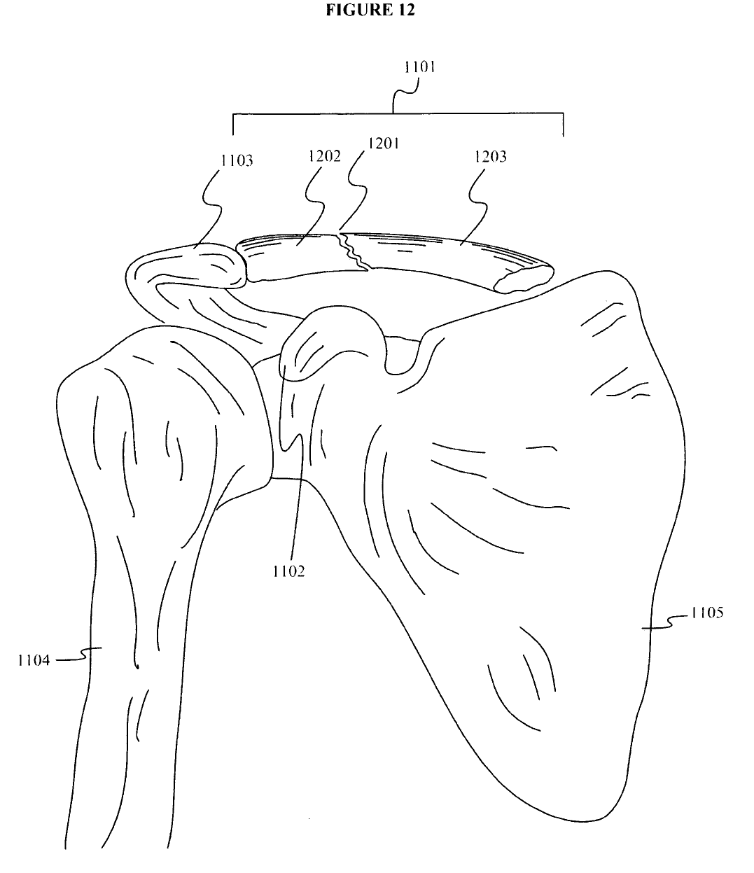

FIG. 12 is a partial perspective view of the bones of the shoulder anatomy illustrating a clavicle 1101 with a fracture 1201. The clavicle 1101 has medial 1203 and lateral 1202 portions relative to the fracture 1201. Also depicted in the illustration are the acromion 1103, the body of the scapula 1105, and the humerus 1104. The coracoid process 1102 extends anteriorly from the scapula 1105. As the illustration shows, the coracoid process 1102 is located at least partially beneath the clavicle 1101.

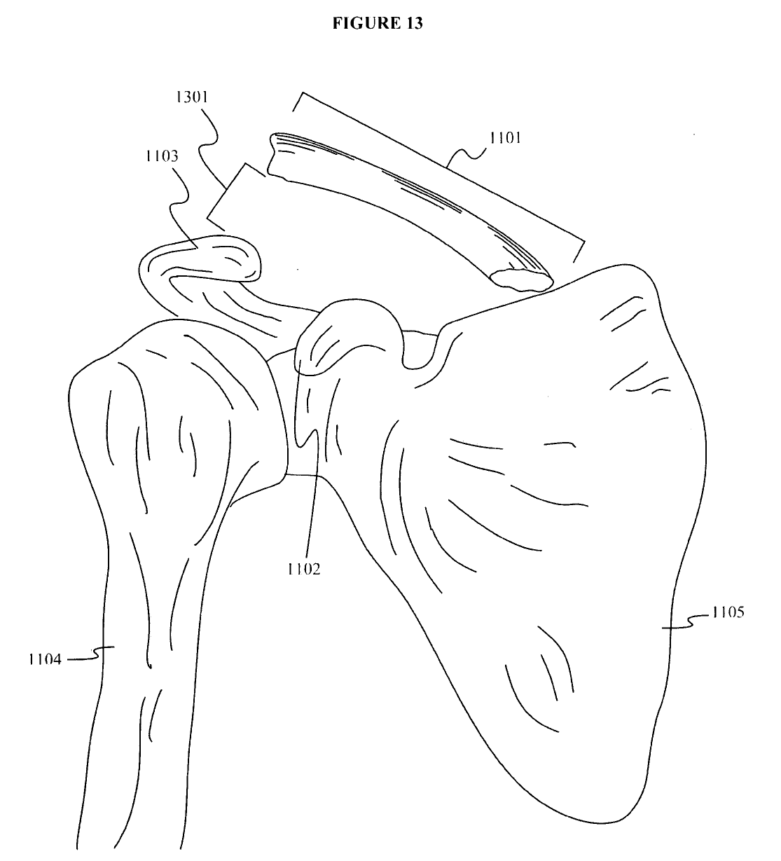

FIG. 13 is a partial perspective view of the bones of the anatomy of a human shoulder depicting a dislocation 1301 of the clavicle 1101. The clavicle 1101 has been displaced 1301 from its normal anatomical position likely due to a disruption of the acromioclavicular and coracoclavicular ligaments. Also depicted in the illustration are the acromion 1103, the body of the scapula 1105, and the humerus 1104. The coracoid process 1102 extends anteriorly from the scapula 1105. As the illustration shows, the coracoid process 1102 is located at least partially beneath the clavicle 1101.

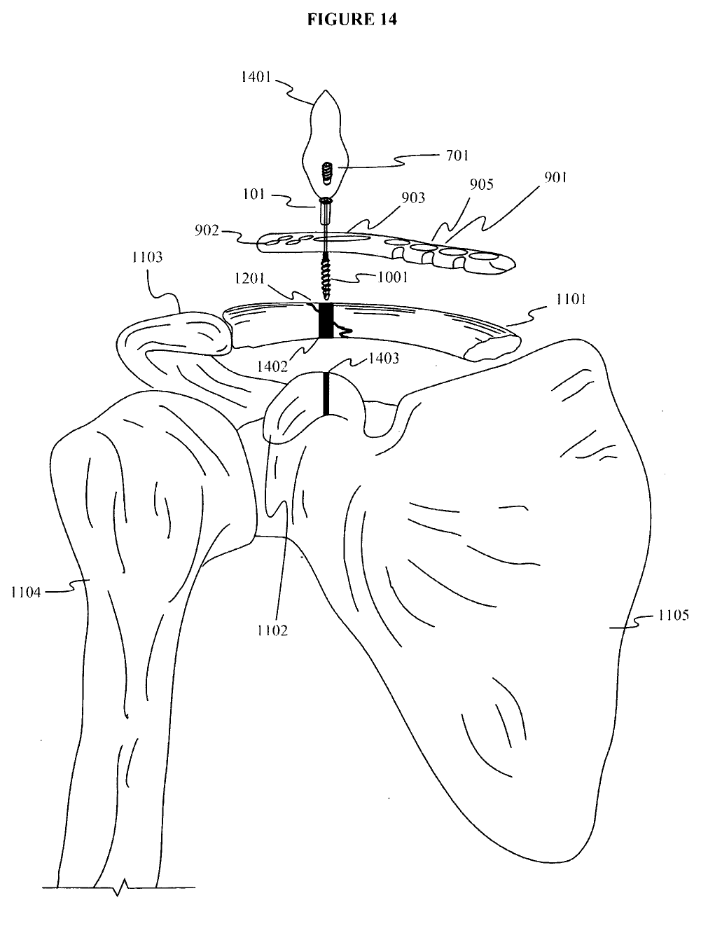

FIG. 14 is a partial perspective view of the bones of the shoulder anatomy illustrating a clavicle 1101 with a fracture 1201 showing an exploded view of the placement of a bone plate 901, threaded sleeve with flange 101, bone anchor 1001, suture or other flexible material 1401, and headless set screw 701. The bone plate 901 is secured to the superior aspect of the clavicle 1101 with a plurality of bone screws inserted into any of a plurality of openings 902 and 905 in the bone plate 901 and into the clavicle 1101. A bone drill is utilized to drill a hole 1402 through The clavicle 1101 from superior to inferior, and a hole 1403 through the coracoid process 1102. A second bone drill is utilized to enlarge the diameter of the hole 1402 through the clavicle 1101 so that the diameter of the hole 1402 is slightly larger than the diameter of the threaded sleeve with flange 101. In this example, an anchor 1001 with an eyelet 1004 that has flexible material 1401, (i.e. suture, cable or other suitable flexible material) pre-attached is passed through the opening in the bone plate 903 and through the hole 1402 through the clavicle 1101 and screwed into the smaller diameter hole 1403 through the coracoid process 1102 so that the anchor 1001 extends completely through the coracoid process and the tip 1003 of the anchor 1001 may protrude minimally on the inferior aspect of the coracoid process 1102. The flexible material 1401 is retrieved through the hole 1402 through the clavicle 1101 and through the opening 903 in the bone the bone plate 901. The flexible material 1401 is passed through the threaded sleeve with flange 101 and the threaded sleeve with flange 101 is then seated in the opening 903 in the proprietary bone plate 901 so that the shaft of the sleeve 104 passes through the hole 1402 through the clavicle 1101. When the threaded sleeve with flange 101 is in place, the flange 102 of the threaded sleeve with flange 101 rests on the recessed lip 904 of the opening 903 in the bone plate 901 and the recessed lip 904 prevents the threaded sleeve with flange 101 from falling through the bone plate 901 and provides a solid surface against which forces can be applied to accomplish the fracture 1201 reduction and aperture fixation. Tension is applied to the flexible material 1401 until the fracture 1201 is reduced and the normal coracoclavicular anatomy is restored. While maintaining tension on the flexible material 1401, the headless set screw 701 is inserted into the threaded sleeve with flange 101 and threaded onto the threads 201 within the shaft of the sleeve 104 until the smooth rounded tip 703 and 704 of the headless set screw 701 meets the smooth tapered surface 202 of the lower portion of the threaded sleeve with flange 101 securing the flexible material 1401 in a compression fit between the rounded tip 703 and 704 of the bullet-shaped headless set screw 701 and the tapered surface 202 of the lower portion of the threaded sleeve with flange 101.

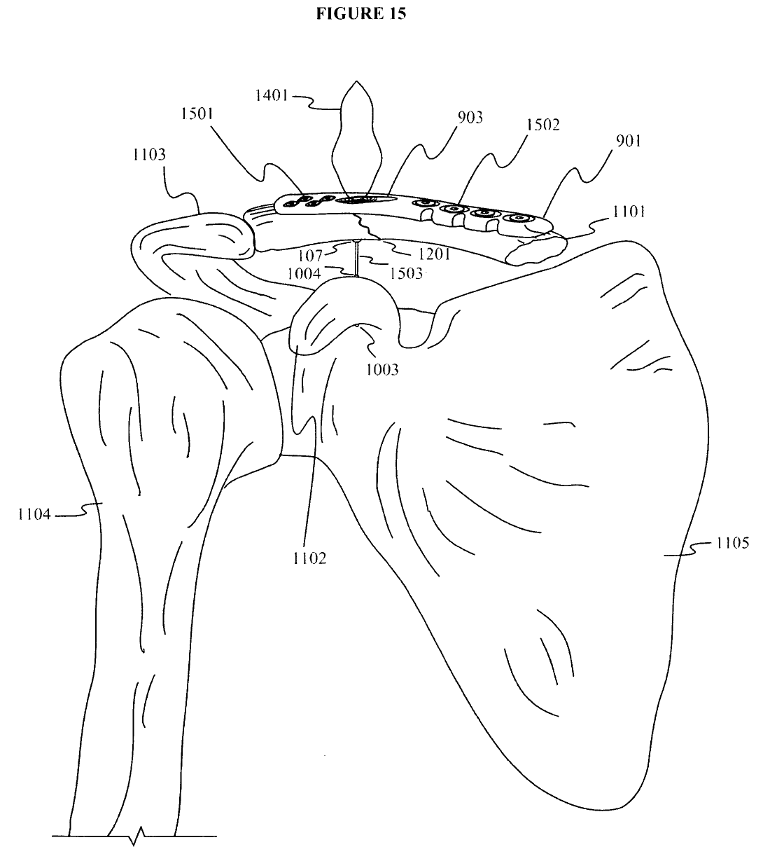

FIG. 15 is a partial perspective view of the bones of the shoulder anatomy illustrating a clavicle 1101 with a fracture 1201 showing the tensioned placement of a bone plate 901, threaded sleeve with flange 101, bone anchor 1001, suture or other flexible material 1401, and headless set screw 701. The bone plate 901 has been secured to the superior aspect of the clavicle 1101 with a plurality of bone screws 1501 and 1502 inserted into any of a plurality of openings 902 and 905 in openings 902 and 905 in the bone plate 901 and into the clavicle 1101. The anchor 1001 with eyelet 1004 that has at least one flexible material 1401, (i.e. suture, cable or other suitable flexible material) pre-attached has been screwed into the hole 1403 through the coracoid process 1102 so that the anchor 1001 extends completely through the coracoid process and the tip 1003 of the anchor 1001 protrudes minimally on the inferior aspect of the coracoid process 1102. The flexible material 1401 has been retrieved through the hole 1402 through the clavicle 1101 and through the opening 903 in the bone plate 901. The flexible material 1401 has been passed through the threaded sleeve with flange 101 and the threaded sleeve with flange 101 has been then seated in the opening 903 in the proprietary bone plate 901 and rests atop the recessed lip 904. The recessed lip 904 prevents the threaded sleeve with flange 101 from falling through the bone plate 901 and provides a solid surface against which forces can be applied to accomplish the fracture 1201 reduction and aperture fixation. Tension has been applied to the flexible material 1401 and the fracture 1201 has been reduced and the normal coracoclavicular anatomy has been restored. While maintaining tension on the flexible material 1401, the headless set screw 701 has been inserted into the threaded sleeve with flange 101 and threaded onto the threads 201 within the shaft of the sleeve 104 until the smooth rounded tip 703 and 704 of the headless set screw 701 met the smooth tapered surface 202 of the lower portion of the threaded sleeve with flange 101 securing the flexible material 1401 in a compression fit between the rounded tip 703 and 704 of the bullet-shaped headless set screw 701 and the tapered surface 202 of the lower portion of the threaded sleeve with flange 101. With the headless set screw 701 in place the tension is maintained between the coracoid process 1102 and the clavicle 1101 (as can be seen by the taut flexible be seen by the taut flexible material 1503 in the figure). At this point, the surgeon has the option to simply cut away the excess flexible material 1401, or to tie a knot in the excess flexible material 1401 above the proximal end of the headless set screw 701. When the surgeon chooses to tie a knot the knot can be recessed within the sleeve.

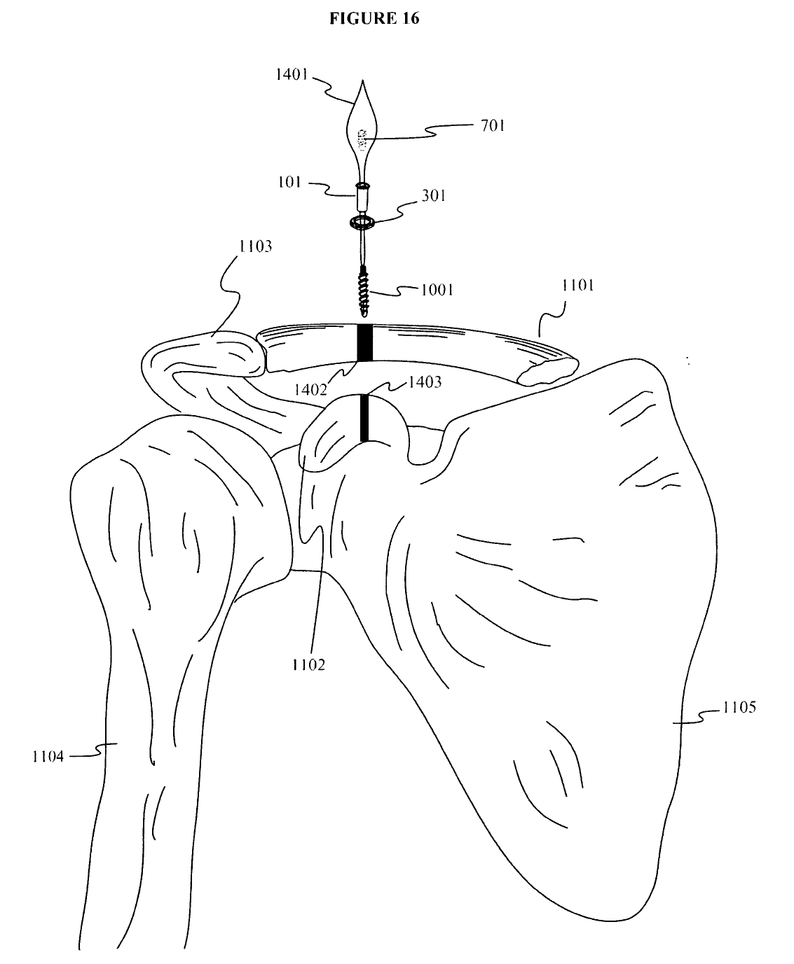

FIG. 16 is a partial perspective view of the bones of the shoulder anatomy illustrating the repair of a high-grade acromioclavicular joint separation with the clavicle 1101 displaced. The clavicle 1101 has already been realigned and the coracoclavicular and acromioclavicular relationships have been restored. The illustration shows the exploded view of a threaded sleeve with flange 101, an extended washer 301, a bone anchor 1001, suture or other flexible material 1401, and headless set screw 701. A bone drill is utilized to drill a hole 1402 through the clavicle 1101 from superior to inferior, and a hole 1403 through the coracoid process 1102. A second bone drill is utilized to enlarge the diameter of the hole 1402 through the clavicle 1101 so that the diameter of the hole 1402 is slightly larger than the diameter of the threaded sleeve with flange 101. In this example, an anchor 1001 with an eyelet 1004 that has flexible material 1401, (i.e. suture, cable or other suitable flexible material) pre-attached is passed through the hole 1402 through the clavicle 1101 and screwed into the smaller diameter hole 1403 through the coracoid process 1102 so that the anchor 1001 extends completely through the coracoid process and the tip 1003 of the anchor 1001 may protrude minimally on the inferior aspect of the coracoid process 1102. The flexible material 1401 is retrieved through the hole 1402 through the clavicle 1101. The flexible, material 1401 is passed through the extended washer 301 and then through the threaded sleeve with flange 101 and the threaded sleeve with flange 101 is then seated on the recessed lip 305 in the opening in the washer 301 so that the flat sides 105 of the flange 102 of the threaded sleeve with flange 101 seats securely against the flat sides 304 of the opening in the extended washer 301. The shaft of the sleeve 104 then passes through the hole 1402 through the clavicle 1101 so that the washer 301 rests on the surface of the clavicle 1101. The recessed lip 305 in the extended washer 301 prevents the threaded sleeve with flange 101 from falling through the extended washer 301 and provides a solid surface against which forces can be applied to accomplish the anatomical reduction and aperture fixation. Tension is applied to the flexible material 1401 until the normal coracoclavicular anatomy is restored. While maintaining tension on the flexible material 1401, the headless set screw 701 is inserted into the threaded sleeve with flange 101 and threaded onto the threads 201 within the shaft of the sleeve 104 until the smooth rounded tip 703 and 704 of the headless set screw 701 meets the smooth tapered surface 202 of the lower portion of the threaded sleeve with flange 101 securing the flexible material 1401 in a compression fit between the rounded tip 703 and 704 of the bullet-shaped headless set screw 701 and the tapered surface 202 of the lower portion of the threaded sleeve with flange 101.

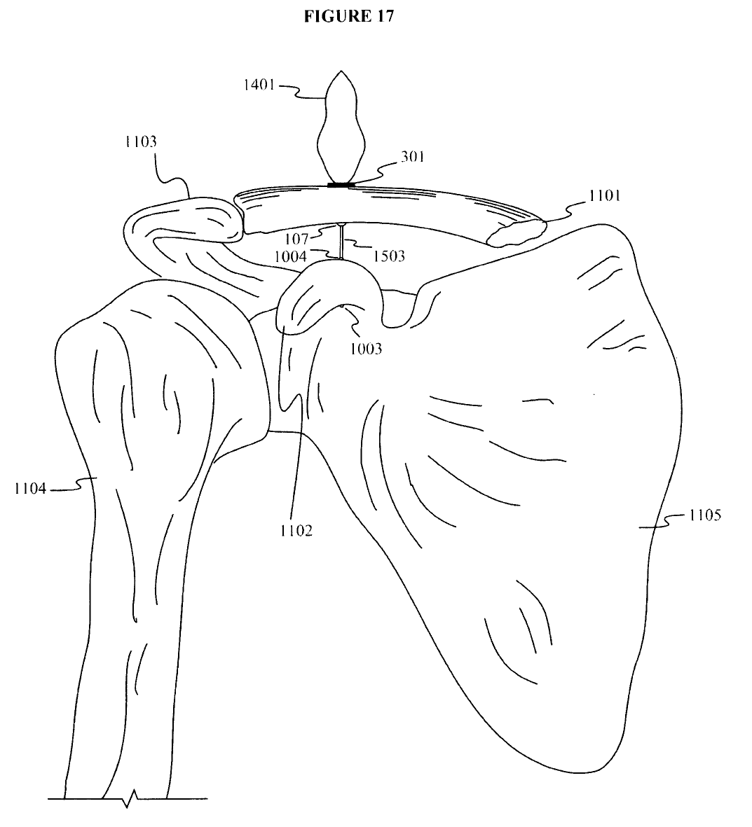

FIG. 17 is a partial perspective view of the bones of the shoulder anatomy illustrating the repair of a high-grade acromioclavicular joint separation with the clavicle 1101 displaced. The clavicle 1101 has already been realigned and the coracoclavicular and acromioclavicular relationships have been restored. The illustration shows the tensioned placement of a threaded sleeve with flange 101, an extended washer 301, a bone anchor 1001, suture or other flexible material 1401, and headless set screw 701. The anchor 1001 with eyelet 1004 that has flexible material 1401, (i.e. suture, cable or other suitable flexible material) pre-attached has been screwed into the hole 1403 through the coracoid process 1102 so that the anchor 1001 extends completely through the coracoid process and the tip 1003 of the anchor 1001 protrudes minimally on the inferior aspect of the coracoid process 1102. The flexible material 1401 has been retrieved through the hole 1402 through the clavicle 1101. The flexible material 1401 has been passed through the extended washer 301 and the threaded sleeve with flange 101 and the threaded sleeve with flange 101 has been seated on the recessed lip 305 in the opening in the washer 301 so that the flat sides 105 of the flange 102 of the threaded sleeve with flange 101 seat securely against the flat sides 304 of the opening in the extended washer 301. The shaft of the sleeve 104 has been passed through the hole 1402 through the clavicle 1101 so that the washer 301 rests on the surface of the clavicle 1101. The recessed lip 305 in the extended washer 301 prevents the threaded sleeve with flange 101 from falling through the extended washer 301 and provides a solid surface against which forces can be applied to accomplish the anatomical reduction and aperture fixation. Tension has been applied to the flexible material 1401 and the normal coracoclavicular anatomy has been restored. The headless set screw 701 has been inserted into the threaded sleeve with flange 101 and threaded onto the threads 201 within the shaft of the sleeve 104 until the smooth rounded tip 703 and 704 of the headless set screw 701 met the smooth tapered surface 202 of the lower portion of the threaded sleeve with flange 101 securing the flexible material 1401 in a compression fit between the rounded tip 703 and 704 of the bullet-shaped headless set screw 701 and the tapered surface 202 of the lower portion of the threaded sleeve with flange 101.

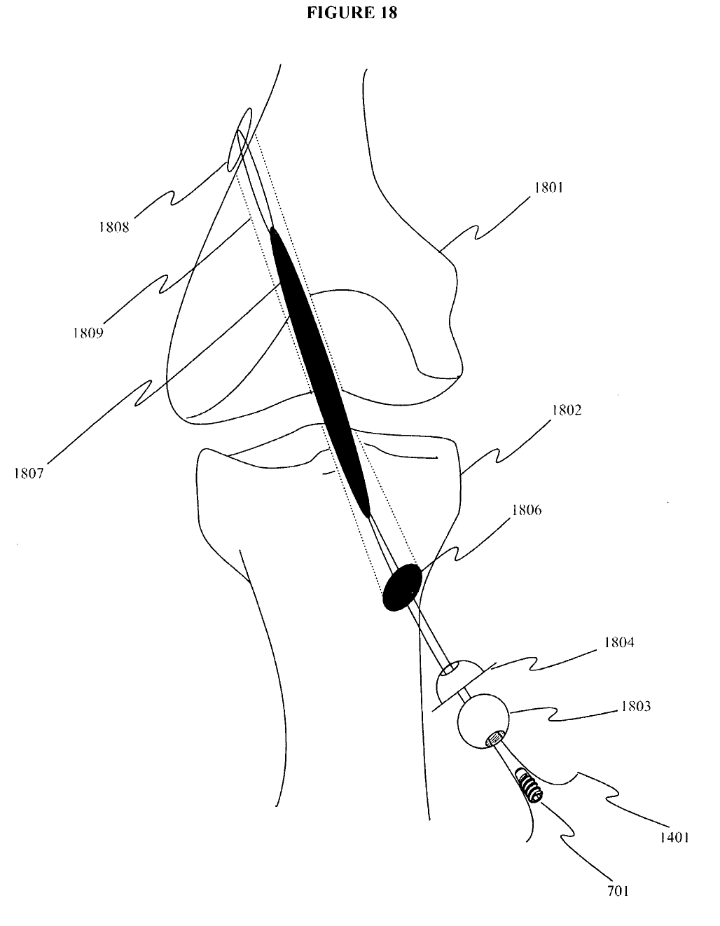

FIG. 18 is a partial perspective view of the bones of the knee anatomy illustrating reconstruction of an anterior cruciate ligament. The illustration shows the exploded view of placement of a flanged washer 1804, a spherical sleeve 1803, a tendon graft 1807, suture or other flexible material 1401, and a headless set screw 701. The flanged washer 1804 has a concave surface designed to hold the spherical sleeve 1803. The depth of the concavity in the flanged washer 1804 is such that when the threaded spherical sleeve 1803 is seated in the flanged washer 1804 the most superficial surface of the spherical sleeve 1803 is situated substantially below the most superficial surface of the flanged washer 1804. The flanged washer 1804 has an opening on its deepest surface with a diameter that is substantially large enough to permit flexible material 1401 that is passed through the opening to subtend a wide range of angles relative to the flat surface of the flanged washer 1804 so that the flexible material does not contact the sides of the opening in the flanged washer 1804. The diameter of the opening in the flanged washer 1804 is smaller than the diameter of the spherical sleeve 1803. A bone drill is utilized to drill bone tunnels 1806 & 1809 through both the tibia 1802 and femur 1801 that will allow the graft to be placed along an anatomic axis of the uninjured ACL. In this type of fixation, the hole through the bone does not go straight through the bone, rather the hole is made at an angle. In this embodiment, the fixation of the graft on the femoral side will be obtained through one of any of the number of commercially available femoral fixation devices 1808. The flexible material on the tibial side of the graft will pass through the tibial tunnel and then be placed through the flanged washer 1804, through the spherical sleeve 1803, tensioned and secured with the headless set screw 701 as previously described. An appropriate tensioning device is utilized to tension the flexible material flexible material 1401. In some embodiments, the spherical sleeve 1803 may have features along its superficial surface to permit the spherical sleeve 1803 to engage an appropriate tensioning device. This feature may also permit the tensioning device to maintain the spherical sleeve 1803 in an optimal orientation within the flanged washer 1804. While maintaining the tension on the flexible material 1401, the headless set screw 701 is inserted into the spherical sleeve 1803 and threaded onto the threads within the spherical sleeve 1803 until the smooth rounded tip 703 & 704 of the headless set screw 701 meets the smooth tapered surface of the lower portion of the interior of the spherical sleeve 1803 securing the flexible material 1401 in a compression fit between the rounded tip 703 and 704 of the bullet-shaped headless set screw 701 and the tapered surface of the lower portion of the interior of the spherical sleeve 1803.

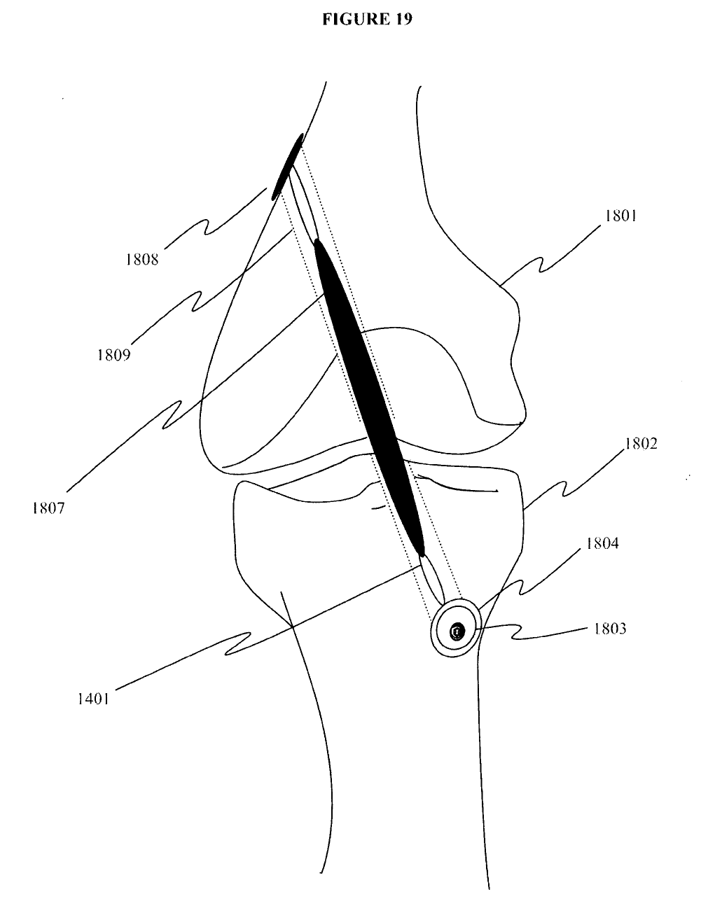

FIG. 19 is a partial perspective view of the bones of the knee anatomy illustrating reconstruction of an anterior cruciate ligament. The illustration shows the tensioned placement of a flanged washer 1804, a spherical sleeve 1803, a tendon graft 1807, suture or other flexible material 1401, and a headless set screw 701. In this embodiment, the fixation of the graft on the femoral side will be obtained through one of any of the number of commercially available femoral fixation devices 1808. The flexible material on the tibial side of the graft was passed through the tibial tunnel and then placed through the flanged washer 1804 and through the spherical sleeve 1803. The spherical sleeve 1803 has been seated in the flanged washer 1804 and the flanged washer 1804 has been seated in the hole 1806 in the tibia 1802. An appropriate tensioning device was utilized to tension the flexible material 1401. While maintaining the tension on the flexible material 1401, the headless set screw 701 was inserted into the spherical sleeve 1803 and threaded sleeve 1803 and threaded onto the threads within the spherical sleeve 1803 until the smooth rounded tip 703 and 704 of the headless set screw 701 met the smooth tapered surface of the lower portion of the interior of the spherical sleeve 1803 securing the flexible material 1401 in a compression fit between the rounded tip 703 and 704 of the bullet-shaped headless set screw 701 and the tapered surface of the lower portion of the interior of the spherical sleeve 1803. As shown in this illustration the spherical sleeve 1803 once seated in the flanged washer 1804 is situated substantially below the most superficial surface of the flanged washer 1804.

The present application is directed toward a fastening device for securing a flexible material (e.g. suture) with knotless fixation through the use of a tapered sleeve and tapered, headless screw.

In general the method includes forming a hole through a bone. Passing a fastening device with a flexible material pre-attached through the hole through the bone and securing the fastening device to a tissue on the opposite side of the bone; passing the free end of the flexible material through a bone plate or extended washer and through a threaded sleeve with flange; positioning the threaded sleeve with flange through the hole and a bone plate or extended washer combination so that the flanged end of the threaded sleeve rests on a recessed shelf within the interior perimeter of the bone plate or washer and the bottom surface of the bone plate or extended washer rests on the surface of the bone; tensioning the suture or other flexible material to restore normal anatomical interval; inserting a bullet-shaped headless set screw into the threaded sleeve with flange and thread the headless set screw onto the threads of the threaded sleeve with flange until the tensioned flexible material is held securely between the bullet-shaped tip of the headless set-tip of the headless set-screw and the tapered lower portion of the threaded sleeve with flange.

The steps of the techniques described can be performed in a variety of different orders. In some instances, one or more of the steps described herein may be discarded completely.

Other implementations are within the scope of the claims.

What is claimed is:

1. A knotless aperture fixation system for fractures and tissue repair comprising:
a cylindrical sleeve having a first proximal end with a substantially round flange extending perpendicular out from the sleeve, a second distal end with smooth rounded edges, an internal diameter with screw threading beginning at the flanged end of the sleeve and extending into the sleeve approximately two-thirds of the length of the interior of the sleeve where the threading stops and the sleeve narrows to a smooth beveled surface ending with smooth rounded edges at the distal end;
a headless set screw having a diameter smaller than that of the threaded portion of the internal diameter of the sleeve, an exterior screw threading extending approximately two-thirds of the length of the screw (from proximal to distal) that leads to a smooth, non-threaded, bullet-shaped tip at the distal end of the headless set screw, and a means of engagement suitable for driving insertion of the headless set screw into the flanged sleeve so as to create a compression fit against a flexible material passed through the sleeve and between the smooth bullet-shaped tip of the headless set screw and the smooth, tapered distal end of the interior diameter of the sleeve;
wherein, the fit between the sleeve and the headless set screw is engaged but loose when the set screw is threaded into the sleeve with no material between the interior of the sleeve and the headless set screw, but which fit becomes firm and tight when a flexible material has been first passed through the sleeve and the set screw is then fully threaded into the sleeve creating a compression fit between the smooth tapered tip of the set screw and the smooth tapered surface of the distal end of the sleeve.

2. The system of claim 1 wherein the internal screw threading of the sleeve and the exterior screw threading of the headless set screw are double threaded to facilitate quicker engagement of the threads and to permit faster insertion of the headless set screw with fewer rotations required.

3. The system of claim 1 wherein the sleeve and headless set screw are composed of titanium.

4. The system of claim 1 wherein the sleeve and headless set screw are composed of PEEK.

5. The system of claim 1 wherein the sleeve and headless set screw are composed of stainless steel.

6. The system of claim 1 wherein the sleeve and headless set screw are composed of another biocompatible material.

7. The system of claim 1 wherein the engaging means is a socket cavity formed in the superior surface of said set screw.

8. The system of claim 1 wherein the engaging means is a raised feature located on the superior surface of said set screw.

9. The system of claim 1 wherein the substantially round flange of the sleeve has two straight edges parallel to and opposite each other.

10. The system of claim 9 wherein the upper portion of the sleeve has two straight edges parallel to and opposite each other and located immediately beneath the substantially round flange so that the straight edges of the upper portion of the sleeve are aligned with the straight edges of the substantially round flange.

11. A knotless aperture fixation system for tissue repair comprising:
a substantially round washer having an outer perimeter that is flat and smooth, a bowl-shaped inner perimeter and a substantially round opening at the center of the inner perimeter;
a substantially round, spherical sleeve having a first proximal opening with a diameter larger than the diameter of a second distal opening wherein the distal opening has a smooth rounded edge, an internal diameter with screw threading beginning at the proximal opening of the sleeve and extending into the sleeve approximately two-thirds of the length of the interior of the sleeve where the threading stops and the sleeve narrows to a smooth beveled surface ending at the smooth rounded edges at the distal opening;
a headless set screw having a diameter smaller than that of the threaded portion of the internal diameter of the sleeve, an exterior screw threading extending approximately two-thirds of the length of the screw (from proximal to distal) that leads to a smooth, non-threaded, bullet-shaped tip at the distal end of the headless set screw, and a means of engagement suitable for driving insertion of the headless set screw into the spherical sleeve so as to create a compression fit against a flexible material passed through the sleeve and between the smooth bullet-shaped tip of the headless set screw and the smooth, tapered distal end of the interior diameter of the sleeve;
wherein, the fit between the sleeve and the headless set screw is engaged but loose when the set screw is threaded into the sleeve with no material between the interior of the sleeve and the headless set screw, but which fit becomes firm and tight when a flexible material has been first passed through the sleeve and the set screw is then fully threaded into the sleeve creating a compression fit between the smooth tapered tip of the set screw and the smooth tapered surface of the distal end of the sleeve.

12. The system of claim 11 wherein the internal screw threading of the sleeve and the exterior screw threading of the headless set screw are double threaded to facilitate quicker engagement of the threads and to permit faster insertion of the headless set screw with fewer rotations required.

13. The system of claim 11 wherein the washer, sleeve and headless set screw are composed of titanium.

14. The system of claim 11 wherein the washer, sleeve and headless set screw are composed of PEEK.

15. The system of claim 11 wherein the washer, sleeve and headless set screw are composed of stainless steel.

16. The system of claim 11 wherein the washer, sleeve and headless set screw are composed of another biocompatible material.

17. The system of claim 11, wherein the engaging means is a socket cavity formed in the superior surface of said set screw.

18. The system of claim 11 wherein the engaging means is a raised feature located on the superior surface of said set screw.

19. A method of repairing a fractured bone wherein the system of claim 1 engages with a bone plate to treat a fracture with knotless fixation, the method comprising:
positioning and provisionally or permanently securing a substantially rigid plate containing containing an elongated opening with a recessed shelf around the perimeter of the opening to an aspect of a fractured bone wherein the plate extends away from the fracture on both sides;
forming a hole through the bone;
passing a fastening device with flexible material pre-attached through the hole formed through the bone;
securing the fastening device to a nearby tissue on the opposite side of the bone where the substantially rigid plate is secured;
passing the free end of the flexible material up through the hole through the bone and through the opening in the substantially rigid plate;
passing the free end of the flexible material through the threaded cylindrical sleeve with flange;
inserting the threaded cylindrical sleeve with flange into the opening in the substantially rigid plate so that the flange of the threaded sleeve comes to rest against, and is flush with, the recessed shelf around the perimeter of the opening in the plate with the portion of the sleeve below the flange passing through or substantially through the hole through the bone;
tensioning the flexible material to restore the normal anatomical interval;
maintaining tension on the flexible material;
inserting a bullet-shaped headless set screw into the threaded sleeve with flange so that the flexible material is between the bullet-shaped headless set screw and the interior lining of the threaded sleeve with flange, while continuing to maintain the tension on the flexible material;
tightening the bullet-shaped headless set screw until the smooth rounded tip of the bullet-shaped headless set screw engages and secures the flexible material between the smooth rounded tip of the bullet-shaped headless set screw and the smooth tapered lower portion of the interior of the threaded sleeve with flange.

20. The method of claim 19 wherein the hole through the bone aligns with an elongated opening with recessed shelf in the substantially rigid plate;

21. The Method of claim 19 wherein the flexible material passed through the fastening device is suture.

22. The method of claim 19 wherein the flexible material passed through the fastening device is cable.

23. The method of claim 19 wherein the flexible material is doubled over at least once to create a plurality of loops of flexible material as well as a plurality of free ends of flexible material.

24. The method of claim 19 wherein the fastening device is an anchor with an eyelet on its proximal end to facilitate attachment of the flexible material.

25. The method of claim 19 wherein the fastening device is a button through which the flexible material passes.

26. The method of claim 25 wherein after the button has been deployed, a second non-threaded cylindrical sleeve with flange is placed on the superior surface of the bone through which the button has been passed in order to provide a smooth channel and avoid abrasion of the flexible material.

27. The method of claim 26 wherein the second non-threaded cylindrical sleeve with flange is composed of a suitable biocompatible material.

28. The method of claim 19 above wherein the headless set screw is tightened utilizing a torque limiting device that tightens the set screw to the appropriate torque/force to ensure maximum strength of the flexible material and sleeve construct.

29. The method of claim 19 wherein the fractured bone is a fractured clavicle and the nearby tissue is the coracoid process.

30. A method of repairing tissue with a knotless fixation wherein the system of claim 1 engages with an extended washer that has a recessed shelf around the inner perimeter of the opening in the washer, the method comprising:
forming a hole through a bone;
passing a fastening device with a flexible material pre-attached through hole formed through the bone;
securing the fastening device to a nearby tissue on the opposite side of the bone through which the fastening device has been passed;
passing the free end of the flexible material up through the hole through the bone;
passing the free end of the flexible material through an extended washer;
passing the free end of the flexible material through the threaded cylindrical sleeve with flange;
inserting the threaded cylindrical sleeve with flange into the opening in the extended washer so that the flange of the threaded sleeve comes to rest against, and is flush with, the recessed shelf around the perimeter of the opening in the extended washer;
inserting the threaded cylindrical sleeve into the hole through the bone so that the bottom of the extended washer rests on the surface of the bone;
tensioning the flexible material to restore the normal anatomical interval;
maintaining tension on the flexible material;
inserting a bullet-shaped headless set screw into the threaded sleeve with flange so that the flexible material is between the bullet-shaped headless set screw and the interior lining of the threaded sleeve with flange, while continuing to maintain the tension on the flexible material;
tightening the bullet-shaped headless set screw until the smooth rounded tip of the bullet-shaped headless set screw engages and secures the flexible material between the smooth rounded tip of the bullet-shaped headless set screw and the smooth tapered lower portion of the interior of the threaded sleeve with flange.

31. The Method of claim 30 wherein the flexible material passed through the fastening device is suture.

32. The method of claim 30 wherein the flexible material passed through the fastening device is cable.

33. The method of claim 30 wherein the flexible material is doubled over at least once to create a plurality of loops of flexible material as well as a plurality of free ends of flexible material.

34. The method of claim 30 wherein the fastening device is an anchor with an eyelet on its proximal end to facilitate attachment of the flexible material.

35. The method of claim 30 wherein the fastening device is a button through which the flexible material passes.

36. The method of claim 30 wherein the fastening device is suture or other flexible material woven through a tendon and is already located on the opposite side of the bone and the free end of the flexible material is passed up through the hole through the bone.

37. The method of claim 30 above wherein the headless set screw is tightened utilizing a torque limiting device that tightens the set screw to the appropriate torque/force to ensure maximum strength of the flexible material and sleeve construct.

38. The method of claim 30 above wherein no washer is used but instead the sleeve with flange sits directly on the bone.

39. The method of claim 30 wherein the tissue injury being treated is a rupture of the coracoclavicular and acromioclavicular ligaments, and the nearby tissue described is the coracoid process.

40. The method of claim 30 wherein the tissue injury being reconstructed is a rupture of the anterior cruciate ligament, and the nearby tissue described is the graft being utilized to reconstruct the anterior cruciate ligament.

41. A method of repairing tissue with a knotless fixation wherein the system of claim 11 is utilized, the method comprising:
forming a hole through a bone;
passing a fastening device with a flexible material pre-attached through the hole formed through the bone;
securing the fastening device to a nearby tissue on the opposite side of the bone through which the fastening device has been passed;
passing the free end of the flexible material up through the hole through the bone;
passing the free end of the flexible material through the opening in the flanged washer and then through the spherical sleeve;
placing the flanged washer against the hole through the bone so that the bowl-shaped underside of the washer rests inside the hole through the bone;
placing the spherical sleeve into the flanged washer so that the distal opening of the sleeve is aligned with the opening in the flanged washer;
rotating the spherical sleeve within the washer to obtain the ideal alignment for tensioning of the flexible material;
tensioning the flexible material to restore the normal anatomical interval;
maintaining tension on the flexible material;
inserting a bullet-shaped headless set screw into the spherical sleeve so that the flexible material is between the bullet-shaped headless set screw and the interior lining of the spherical sleeve, while continuing to maintain the tension on the flexible material;
tightening the bullet-shaped headless set screw until the smooth rounded tip of the bullet-shaped headless set screw engages and secures the flexible material between the smooth rounded tip of the bullet-shaped headless set screw and the smooth tapered lower portion of the interior of the spherical sleeve.

42. An extended washer for use in tissue repair, the extended washer comprising:
a substantially round outer perimeter;
a first substantially round inner perimeter of a diameter smaller than that of the outer perimeter;
a second substantially round inner perimeter of a diameter smaller than that of the first substantially round inner perimeter;
a substantially round recessed shelf situated below the superior surface of the outer perimeter and extending inward from the first substantially round inner perimeter to the second substantially round inner perimeter upon which the flange of a cylindrical sleeve with flange may rest when the cylindrical sleeve is passed through the second substantially round inner perimeter.

43. The extended washer of claim 42 wherein the outer perimeter has two flattened sides parallel to and opposite each other.

44. The extended washer of claim 42 wherein the outer perimeter has two pairs of flattened sides parallel to and opposite each other.

45. The extended washer of claim 42 wherein the first inner perimeter has two flattened sides parallel to and opposite each other that correspond to the flange of a cylindrical sleeve with flange that possesses a substantially round flange with two flattened sides parallel to and opposite and opposite each other and create a lock and key type of fit when the flange of the cylindrical sleeve and extended washer engage together.

46. The extended washer of claim 42 wherein the first inner perimeter has two pairs of flattened sides parallel to and opposite each other that correspond to the flange of a cylindrical sleeve with flange that possesses a substantially round flange with two pairs of flattened sides parallel to and opposite each other and create a lock and key type of fit when the flange of the cylindrical sleeve and extended washer engage together.

47. The extended washer of claim 45 wherein the second inner perimeter has two flattened sides parallel to and opposite each other and the flattened sides of the second inner perimeter are on the same sides of the extended washer as the flattened sides of the first inner perimeter.

48. The extended washer of claim 46 wherein the second inner perimeter has two pairs of flattened sides parallel to and opposite to each other and the flattened sides of the second inner perimeter are on the same sides of the extended washer as the flattened sides of the first inner perimeter.

49. The extended washer of claim 42 wherein the inferior surface of the washer is contoured to correspond to the shape of the bone on which the washer sits.

50. The extended washer of claim 42 wherein the inferior surface of the washer is substantially flat.

51. The extended washer of claim 42 wherein the washer is composed of titanium.

52. The extended washer of claim 42 wherein the washer is composed of PEEK.

53. The extended washer of claim 42 wherein the washer is composed of stainless steel.

54. The extended washer of claim 42 wherein the washer is composed of another biocompatible material.
